(19) United States
(12) Patent Application Publication
(10) Pub. No.: US 2025/0064318 A1
(43) Pub. Date: Feb. 27, 2025

(54) INGESTIBLE CHEMICAL ENERGY HARVESTING SYSTEM WITH EXTENDED LIFETIME

(71) Applicant: Massachusetts Institute of Technology, Cambridge, MA (US)

(72) Inventors: Carlo Giovanni TRAVERSO, Newton, MA (US); Jason Siu Wei Li, Cambridge, MA (US); So-Yoon Yang, Cambridge, MA (US)

(73) Assignee: Massachusetts Institute of Technology, Cambridge, MA (US)

(21) Appl. No.: 18/724,935

(22) PCT Filed: Dec. 28, 2022
(86) PCT No.: PCT/US2022/082492

§ 371 Date: Jun. 27, 2024

Related U.S. Application Data
(60) Provisional application No. 63/294,902, filed on Dec. 30, 2021.

(51) Int. Cl. A61B 5/00; A61B 5/07

(52) U.S. Cl. CPC A61B 5/0031; A61B 5/073

(57) ABSTRACT

A device is configured to be administered via an oral route by a subject. The device includes an anode, a seal disposed on the anode, and a cathode. When exposed to a liquid or a hydrogel, an exposed surface of the anode undergoes galvanic oxidation dissolution to provide DC power to the device. As the exposed surface of the anode undergoes galvanic oxidation dissolution, the seal incrementally detaches from the anode, and a substantially constant surface area of the exposed surface is maintained.

### CROSS-REFERENCE TO RELATED APPLICATION(S)

This application claims the priority benefit, under 35 U.S.C. 119 (e), of U.S. Application No. 63/294,902, filed Dec. 30, 2021, which is incorporated herein by reference in its entirety.

### BACKGROUND

Chronic diseases and conditions are increasingly prevalent in human adults. As human life expectancy increases, the prevalence of chronic diseases and conditions also increases. Chronic diseases and conditions are persistent, continuous, or otherwise long-lasting in their effects, typically last for more than three months. Chronic diseases and conditions include heart disease, cancer, diabetes, stroke, and arthritis. They often require ongoing medical intervention and can also limit one's ability to participate in typical activities during daily life.

People are increasingly using biomedical electronic devices to help manage chronic diseases and conditions. There are several types of biomedical electronic devices. Some biomedical electronic devices are diagnostic devices that diagnose a disease or condition. Other types of biomedical electronic devices are therapeutic devices that treat diseases and conditions. Other types of biomedical electronic device are closed-loop systems that include both diagnostic and therapeutic components.

There is an increasing number of applications in which biomedical electronic devices can be used. Advances have increased the number of features and functionalities that are available in these devices. For example, advances in wireless communication allow medical devices to transmit data to external systems while untethered inside the human body. With these and other advances, the complexity of biomedical electronic device technology has increased.

The increased complexity of biomedical electronic device technology has resulted in biomedical devices with higher energy demands. Increasing energy density is a challenge in implantable and ingestible biomedical devices because of their limited size. Conventional implantable and ingestible biomedical devices use rigid batteries that occupy more than half of the total volume of the device. Conventional ingestible biomedical electronic devices can typically only be powered for up to about 10 hours. Therefore, these conventional devices are not well-suited for monitoring or treating conditions over longer periods of time.

There are several single-use diagnostic and therapeutic ingestible biomedical electronic devices used for short periods of time (e.g., for acute disease applications). These devices have finite energy density power sources that are insufficient for more than a few days of use. Since these devices are intended to be used for a single, short period of time, they are not well suited for treating and managing chronic diseases and conditions.

### SUMMARY

Chemical energy harvesting (CEH) cells were developed with prolonged power generation lifetimes up to several months. These cells have tunable anode dissolution rates that provide tunable rate performance and lifetime. The CEH cells can provide consistent power (e.g., within about 10 μW to about 100 μW of an average power output) over time while still being small enough to be easily ingested. One or more CEH cells can be incorporated into or otherwise coupled with a biomedical electronic device that is orally administered (e.g., ingested or deployed via endoscope), where the CEH cells provide electrical power to the biomedical electronic device.

An embodiment of the invention includes a device configured to be administered via an oral route by a subject. The device includes an anode, a cathode, and a seal. The anode includes a first metal, and the cathode includes a second metal. The seal is disposed on all surfaces of the anode except for a portion constituting an exposed surface of the anode. The cathode is disposed on at least part of the seal and is electrically coupled to the anode. The anode and the cathode are configured to provide DC power to the device when the exposed surface undergoes galvanic oxidation dissolution in at least one liquid or hydrogel. The seal is configured to incrementally detach from the anode when the exposed surface undergoes galvanic oxidation dissolution in the liquid or hydrogel.

The seal may include a plurality of partially overlapping O-rings arranged to form a cylindrical stack having a substantially smooth lateral surface. The O-rings may include a silicone elastomer. Alternatively, or additionally, the seal may include a plurality of O-rings arranged adjacent to each other in a cylindrical stack, with the stack having a gap between each adjacent O-ring and a biodegradable polymer disposed at least partially in the gap between each O-ring. Alternatively, or additionally, the seal may include a biodegradable polymer having a thickness gradient along a surface of the anode such that the seal is thicker along a longitudinal axis of the anode away from the exposed surface. Alternatively, or additionally, the seal may include a biodegradable polymer having a molecular weight gradient along a surface of the anode such that the molecular weight of the seal is higher along a longitudinal axis of the anode away from the exposed surface.

The liquid or hydrogel in which the anode undergoes galvanic oxidative dissolution may be gastric fluid or intestinal fluid. The device may include an ingestible capsule having a cavity. The ingestible capsule may include a microprocessor disposed in the cavity, at least one sensor operably coupled to the microprocessor, at least one actuator operably coupled to the microprocessor and the sensor, and an antenna operably coupled to the microprocessor. The first metal and the second metal may be biocompatible. The first metal may include zinc and the second metal may include a noble metal.

The anode may have a cylindrical shape with a lateral surface, a first base, and a second base. The exposed surface of the anode may constitute the first base. The seal may be disposed on both (a) the lateral surface and (b) the second base. The seal may be configured to incrementally detach from the lateral surface along a longitudinal axis of the anode in a direction from the first base to the second base when the exposed surface undergoes galvanic oxidation dissolution in the liquid and/or hydrogel.

The exposed surface may have a surface area that remains substantially constant during galvanic oxidation dissolution. Once the device is administered, the DC power may be provided for about 8 days to about 60 days.

Another embodiment of the invention includes a system configured to be administered via an oral route by a subject. The system includes a microprocessor, at least one sensor, at least one actuator, an antenna, and a galvanic cell. The one or more sensors are operably coupled to the microprocessor. The one or more actuators are operably coupled to the microprocessor and the sensor. The antenna is operably coupled to the microprocessor. The galvanic cell provides power to the microprocessor, the sensor(s), and the actuator(s). The galvanic cell includes an anode, a seal, and a cathode. The seal is disposed on all surfaces of the anode except for a portion constituting an exposed surface. The cathode is electrically coupled to the anode and disposed on at least part of the seal. The seal is configured to maintain a substantially constant surface area of the exposed surface of the anode when the exposed surface undergoes galvanic oxidation dissolution in the liquid and/or hydrogel. The seal is also configured to maintain a substantially constant distance between the cathode and the exposed surface of the anode when the exposed surface undergoes galvanic oxidation dissolution in the liquid and/or hydrogel. The system may include one, two, or more galvanic cells. The galvanic cells may be electrically coupled in series or in parallel.

Another embodiment of the invention includes a method. The method includes orally administering a device to a subject. The device includes an anode, a seal, and a cathode. The seal is disposed on all surfaces of the anode except for a portion constituting an exposed surface. The cathode is disposed on at least part of the seal and electrically coupled to the anode. The anode and the cathode are configured to provide DC power to the device when the exposed surface undergoes galvanic oxidation dissolution in a liquid in the subject's gastrointestinal tract. The exposed surface has a surface area that remains substantially constant when the exposed surface undergoes galvanic oxidation dissolution. The cathode and the exposed surface are separated by a distance that remains substantially constant when the exposed surface undergoes galvanic oxidation dissolution.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A illustrates electronic components that can be deployed in an orally-administered device.

FIG. 1B is a schematic of a galvanic cell.

FIG. 2A is a schematic of an orally-administered medical device powered by chemical energy harvesting cells.

FIG. 2B is a circuit schematic of the microprocessor block.

FIG. 2C is a photograph of the circuit.

FIG. 2D shows an embodiment of an O-ring in one of the chemical energy harvesting cells shown in FIG. 2A.

FIG. 2E shows additional views of the O-ring shown in FIG. 2D.

FIG. 2F shows other embodiments of an O-ring in one of the chemical energy harvesting cells shown in FIG. 2A.

FIG. 2G shows an energy harvesting cell in one of the chemical energy harvesting cells shown in FIG. 2A.

FIG. 2H shows a picture of an energy harvesting cell.

FIG. 2I shows another orally-administered medical device powered by chemical energy harvesting cells.

FIG. 2J shows another orally-administered medical device powered by chemical energy harvesting cells.

FIG. 2K shows another view of the orally-administered medical device shown in FIG. 2J.

FIG. 3A shows galvanic cells connected in series.

FIG. 3B shows galvanic cells connected in parallel.

FIG. 4 shows another embodiment of a chemical energy harvesting cell.

FIG. 5 shows another embodiment of a chemical energy harvesting cell.

FIG. 6A shows another embodiment of a chemical energy harvesting cell.

FIG. 6B shows another embodiment of a chemical energy harvesting cell.

FIG. 7 shows another embodiment of a chemical energy harvesting cell.

FIG. 8A shows a plot of power output versus exposed anode surface area in a chemical energy harvesting cell.

FIG. 8B shows a plot of power output versus exposed cathode surface area in a chemical energy harvesting cell.

FIG. 9 shows in vitro changes in a chemical energy harvesting cell over time.

FIG. 10 shows in vitro changes in a conventional galvanic cell over time.

FIG. 11 shows power output over time for both a chemical energy harvesting cell and a conventional galvanic cell.

FIG. 12 shows O-ring detachment data over time in a chemical energy harvesting cell.

FIG. 13A shows an experimental setup for testing the chemical energy harvesting cell in vivo.

FIG. 13B shows a plot of power and temperature over time from the chemical energy harvesting cell deployed in vivo in the setup shown in FIG. 13A.

FIG. 13C shows a plot of power and radio signal strength over time from the chemical energy harvesting cell deployed in vivo in the setup shown in FIG. 13A.

FIG. 14A shows an exploded view of another version of a medical device powered by chemical energy harvesting cells.

FIG. 14B shows a perspective view of the medical device in FIG. 14A.

FIG. 15A shows a galvanic cell pH sensor.

FIG. 15B shows data from the pH sensor in FIG. 15A.

FIG. 16A shows another galvanic cell pH sensor.

FIG. 16B shows data from the pH sensor in FIG. 16A.

### DETAILED DESCRIPTION

Diagnostic biomedical electronic devices detect the presence of a disease or condition, and/or monitor the progression of a disease or condition. Diagnostic biomedical electronic devices can also be used to assess the efficacy of treatment through therapeutic drug monitoring or medication adherence monitoring. As an example, diagnostic biomedical electronic devices can be used to monitor diseases including diabetes, cancer, hypertension, heart disease, stroke, respiratory disease, chronic kidney disease, arthritis, and obesity.

Therapeutic biomedical electronic devices treat diseases and can increase medication adherence and treatment efficacy. These devices administer pharmaceutical medicines or other therapeutic interventions such as electrical stimulation of nerves or tissues. In one version of a therapeutic biomedical electronic device, medicines may be administered in a continuous or time-controlled manner using one or more actuators and/or a programmable pump that pumps medicine into the body to maintain analyte concentrations within a targeted therapeutic window. In another version of a therapeutic biomedical electronic device, the device may apply electrical stimulation to tissue and/or nerves to repair neurological dysfunction or to relieve pain. Stimulation may be used in the treatment of several diseases and conditions, including Parkinson's disease, gastroparesis, and chronic pain.

Some biomedical electronic devices are closed-loop systems that include both diagnostic components and therapeutic components, where therapeutic treatment is responsive to diagnostic measurements. Closed-loop systems can provide improved management of chronic diseases and conditions by responding to real-time changes in the body as they happen. One embodiment of a closed-loop system includes diagnostic sensors to detect and/or monitor disease, a central processing unit (e.g., a microprocessor) to analyze the sensor data and adjust treatment, and a treatment unit to administer a medicine or provide another type of therapeutic intervention. Closed-loop systems can be implemented for the treatment of many diseases, including diabetes, cancer, arthritis, pain management, epilepsy, Parkinson's disease, cardiac arrhythmia, and heart disease. For example, type 1 diabetes can be effectively managed using a closed-loop glucose monitoring and insulin pump system. As another example, an implantable closed-loop system can detect food intake and trigger a gastric stimulator that makes a patient feel satiated.

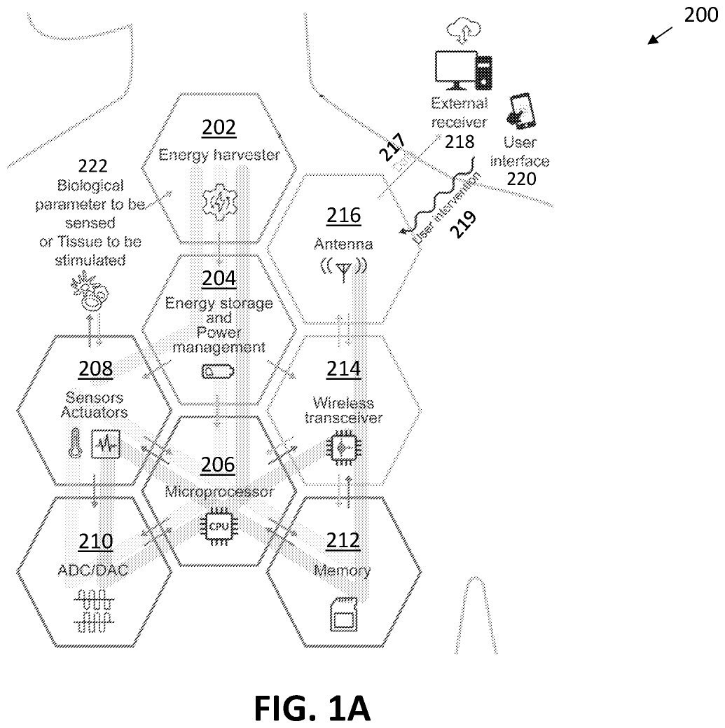

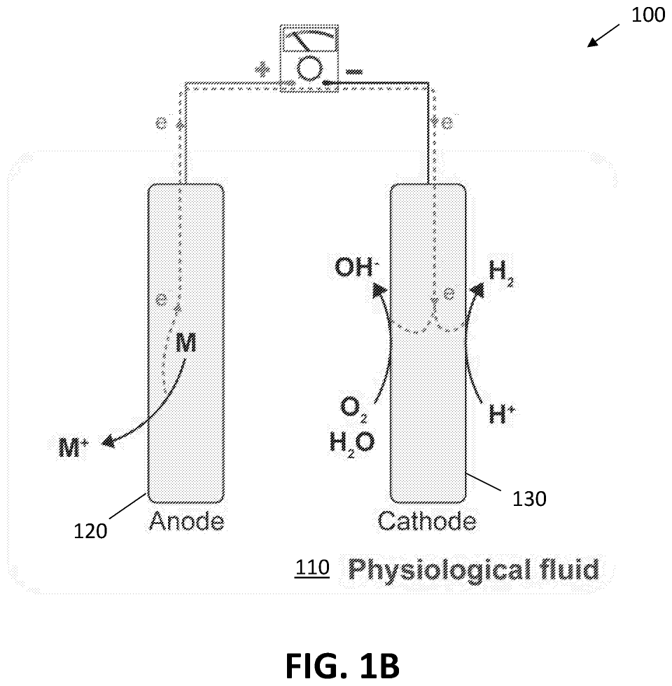

FIG. 1A illustrates several electronic components that can be deployed in an orally-administered medical device 200. These devices include, for example, diagnostic, therapeutic, and/or closed-loop components. Oral administration includes ingestible capsules and delivery via endoscope. The device 200 may be powered by one or more CEH cells 202. The device 200 may include energy storage and power management components 204, a microprocessor 206, sensors and actuators 208, a power converter 210, a data storage component 212, a wireless transceiver 214, and/or an antenna 216. The wireless transceiver 214 and the antenna 216 may transmit data 217 to an external receiver 218 (e.g., a computer or a smart phone), and may receive control signals 219 from a user via user interface 220 on a computer or a smart phone. The sensors and actuators 208 may interface with the biomedical environment to sense external stimuli (e.g., for a diagnostic device) and/or generate medical interventions (e.g., for a therapeutic device). The microprocessor 206 may control the operation of the device 200. The microprocessor 206 provide input/output (I/O) operations, analog and digital signal conversion and processing, peripheral control, memory storage, and/or timing operations. The data storage component 212 may be integrated into the microprocessor 206 or may be a separate component operably coupled to the microprocessor 206. One or more of the electronic components in the device 200 may be electrically coupled to and powered by one or more CEH cells 202.

CEH cells harvest energy from acidic or neutral liquid or hydrogel. As an example, FIG. 1B shows a schematic of a galvanic cell 100 where a physiological fluid 110 acts as the electrolyte. The galvanic cell 100 includes a zinc anode 120 and a gold cathode 130 that are electrically connected. In the physiological fluid, the following chemical reactions occur at the electrodes to generate direct current (DC) power:

| | | |
|---|---|---|
| Anode: | Zn₍ₛ₎ → Zn²⁺_{(aq)} + 2e⁻, | E⁰ = −0.76 V |
| Cathode: | 2H⁺_{(ag)} + 2e⁻ → H_{2(g)}, | E⁰ = 0 V |

Hydrogen gas is produced at the cathode 130. The hydrogen gas may be exhaled via the upper or lower GI tract or absorbed into the blood stream, transported to the lungs, and then exhaled from the lungs. Alternatively, the anode may include aluminum and/or magnesium, and the cathode may include platinum and/or carbon. The anode 120 is ionized and dissolved in the electrolyte 110 as the galvanic cell 100 generates power. The amount of power generated and the lifetime of the galvanic cell 100 are directly related to the size of the anode 120. Conventionally, the size of the anode 120 is constrained by the dimensions of an orally-administered device, so that the amount of power generated and the lifetime of the galvanic cell 100 are similarly constrained. Like ingestible devices with rigid batteries, ingestible devices with CEH galvanic cells are limited to single-use applications with a lifetime of up to about 3 months.

A CEH cell is coupled to an orally-administered biomedical electronic device to provide consistent power to the biomedical electronic device inside of a gastrointestinal (GI) tract for up to several months. The CEH cell can provide DC power inside of the GI tract for about 8 days to about 60 days The CEH cell includes at least one anode with a limited exposed surface area for electrochemical reaction in the body. The CEH cell generates direct current (DC) power when the electrodes are immersed in a hydrogel or a liquid (e.g., a physiological fluid like acidic gastric fluid or intestinal fluid). The anode is ionized and dissolves into the electrolyte as the CEH cell undergoes power generating electrochemical reactions.

The power-generating lifetime of the CEH cell is determined by the dissolution rate of the anode and the mass of the anode. The amount of power output and the lifetime over which the CEH cell provides power is controlled by changing the size of the exposed surface area of the anode in contact with the liquid or hydrogel. The size of the exposed surface area of the anode determines the dissolution rate of the anode. Preferably, the cell provides a substantially constant (e.g., +20% of initial) exposed surface area of the anode and a substantially constant (e.g., +20% of initial) distance between the anode and the cathode to provide a consistent supply of direct current (DC) power for a period of time. As an example, a CEH cell with a diameter of 3.18 mm and a length of 12.5 mm provided power for up to 3 months.

The dissolution rate of the anode is controlled using a seal disposed on all surfaces of the anodes except for at least part of a surface, which defines the exposed surface of the anode. The seal may be made of one or more materials and one or more parts. A larger exposed anode surface area provides a higher current over a shorter lifetime, whereas a smaller exposed anode surface area provides a smaller current over a longer lifetime. As the exposed surface of the anode reacts and dissolves in the liquid (e.g., physiological fluid) or hydrogel, the seal incrementally detaches or dissolves so that the exposed surface area of the anode remains substantially constant (e.g., #20% of initial). The physiological fluid is preferable gastric fluid. Alternatively, the physiological fluid may be fluid in the small or large intestine, or fluid in the subcutaneous space.

The cathode is disposed on at least a part of the seal. An intermediate layer may be positioned between the cathode and the part of the seal, or the cathode may be disposed directly on part of the seal without an intermediate layer, and in either configuration the cathode is disposed on a part of the seal. The intermediate layer may be part of a holder that holds the cathode and the anode in fixed positions. As an example, the holder may be polymer or epoxy.

The cathode is positioned within the CEH cell such that the distance between the cathode and the exposed surface area of the anode remains substantially constant (e.g., +20% of initial) even as the anode reacts and dissolves. For example, the anode may have a cylindrical shape with one base exposed (this base defining the exposed surface of the anode where electrochemical reaction and dissolution occurs). In this example, the cathode is disposed at least partially along the length of the lateral surface of the cylinder so that, as the height of the cylinder decreases due to galvanic oxidation dissolution, the distance between the cathode and the exposed surface of the anode does not substantially change (e.g., #20% of initial). Since the cathode surrounds the cylindrical anode, the distance between the exposed surface of the cylindrical anode and the cathode remains substantially constant (e.g., +20% of initial) as long as the seal does not block the pathway between the anode and the cathode.

The seal disposed on the lateral surface of the anode is a polymer. In an embodiment, the seal is mechanically and chemically stable in the liquid or hydrogel. In this embodiment, the seal incrementally detaches from the surface of the anode as the anode undergoes galvanic oxidation dissolution to maintain the substantially constant (e.g., +20% of initial) exposed anode surface area. In this embodiment, the seal material may include an elastomer (e.g., polydimethylsiloxane (PDMS), another silicone like Ecoflex, thermoplastic polyurethane like Elastollan, or another biocompatible and elastic material) and/or an epoxy.

In another embodiment, the seal dissolves in the liquid or hydrogel. In this embodiment, the seal dissolves at a similar rate (e.g., +20%) to the anode to maintain the substantially constant exposed anode surface area. In this embodiment, the seal material may include a biodegradable polymer (e.g., polycaprolactone (PCL), polyglycolide, polylactic acid, poly lactic-co-glycolic acid (PLGA), polyanhydride, or copolymers thereof). The degradation rate of the seal may be tuned to match the dissolution rate of the anode in the physiological fluid in which the CEH cell is deployed. The degradation rate of the seal may be tuned by tuning the molecular weight, polymer chain termination, ratio of constituent monomers, and thickness of the seal.

In another embodiment, the seal includes multiple components, some stable and some dissolvable. In this embodiment, the dissolvable portions of the seal may dissolve in a controlled manner to incrementally release the mechanically stable portions of the seal in order to maintain the substantially constant exposed anode surface area.

The seal, cathode, and anode may be made, at least in part, of biocompatible materials. For example, the seal may include a biocompatible polymer (e.g., PDMS, PCL, PLGA, Elastollan thermoplastic polyurethane, Ecoflex silicone, polylactic acid, or polyanhydride). The cathode may include a biocompatible metal, carbon (e.g., activated carbon, carbon fiber, or graphite), and/or copper chloride. The biocompatible metal used in the cathode may include a noble metal (e.g., gold, platinum, or silver) or copper. The anode may also be a biocompatible metal (e.g., zinc or magnesium). Since the anode dissolves during the galvanic redox reaction that produces DC current, the anode dissolution rate may be configured to provide an amount of dissolved anode material in the body that is no more than the maximum recommended daily intake (Tolerable Upper Intake Limit, UL) as provided by the Food and Nutrition Board (FNB) at the Institute of Medicine of the National Academies.

The anode has a lower standard electrode potential than the cathode in the electrolyte (here, the liquid or hydrogel in which the cell is immersed). The cell potential, power, and lifetime of the CEH cell depends on the materials used for the anode and the cathode. This is because each metal exhibits a different standard reduction potential and overpotential. For example, the CEH cell with magnesium as an anode and gold as a cathode (Mg/Au cell) yields a theoretical cell potential of 2.38 V, the CEH cell with an aluminum anode and a gold cathode (Al/Au cell) yields a theoretical cell potential of 1.68 V, and a cell with zinc as an anode and gold as a cathode (Zn/Au cell) yields 0.76 V. Thus, the Mg/Au cell generates a higher power than the Zn/Au cell, but the lifetime of the Mg/Au cell is shorter since the dissolution rate of the magnesium anode is higher than that of the zinc anode.

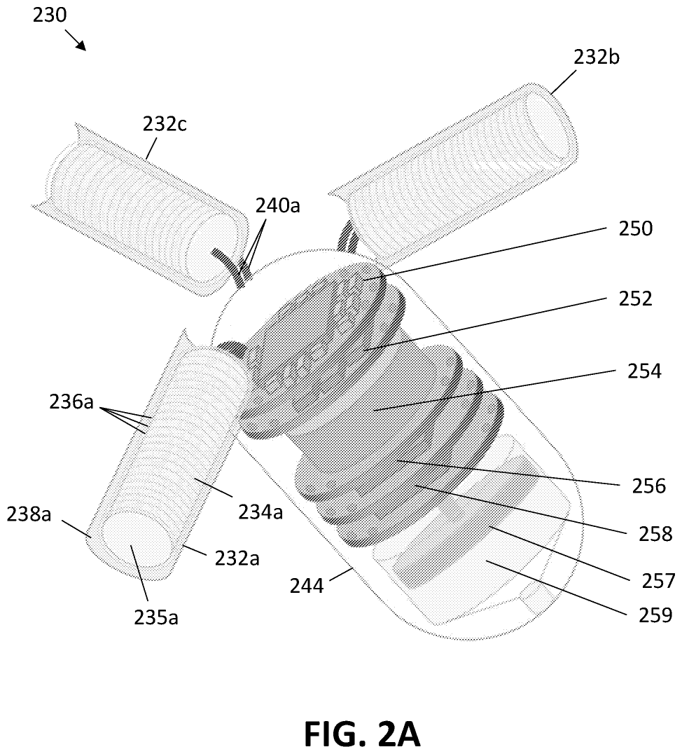

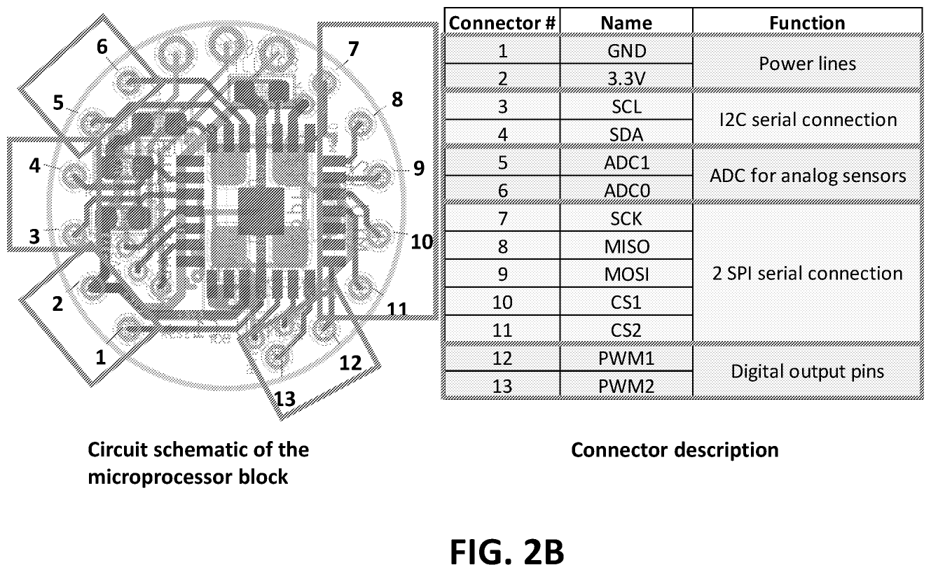

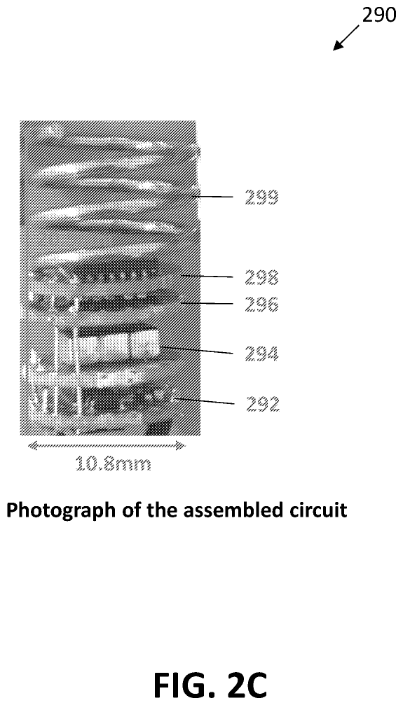

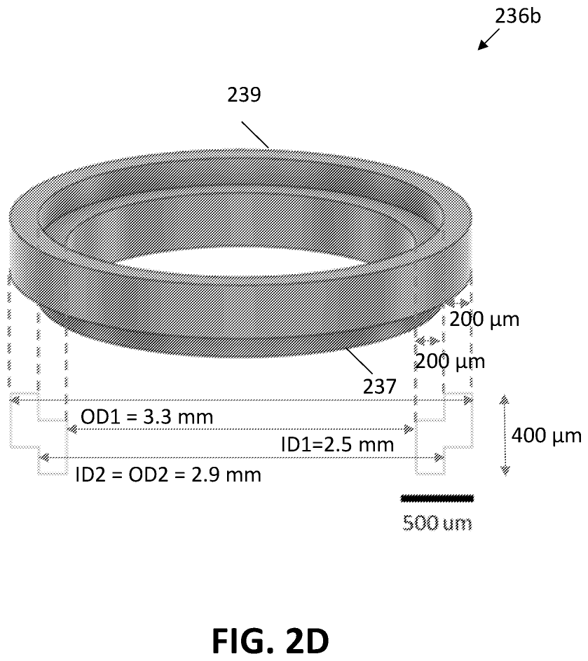

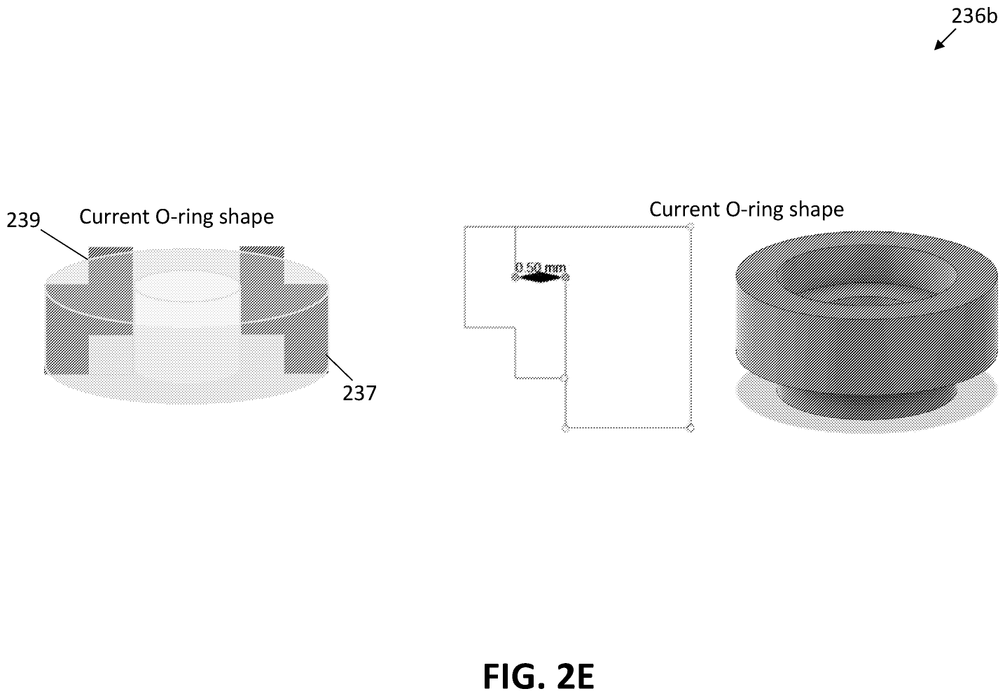

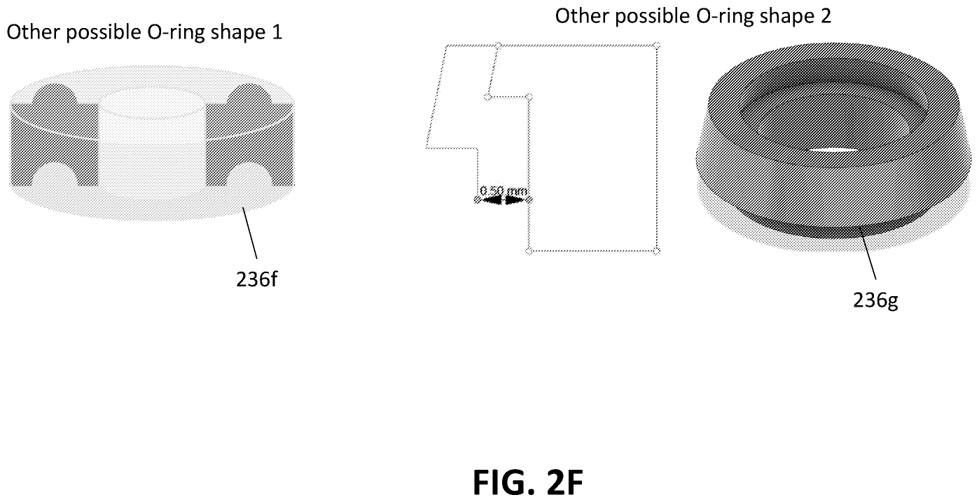

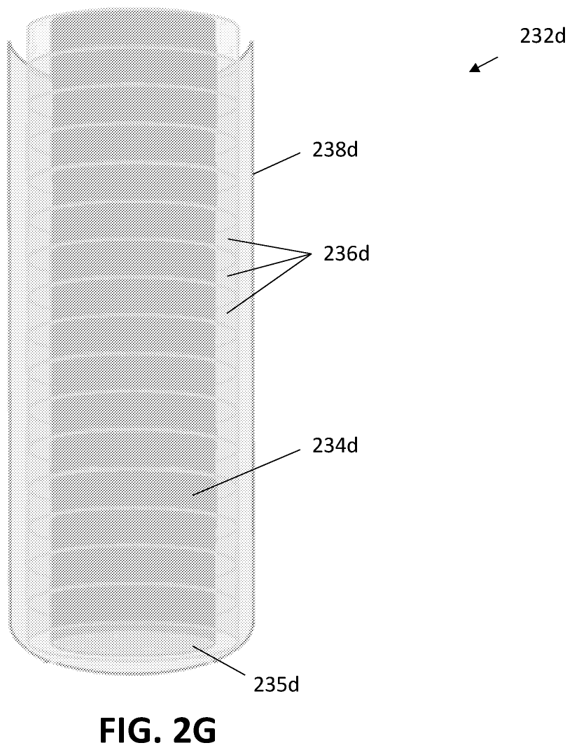

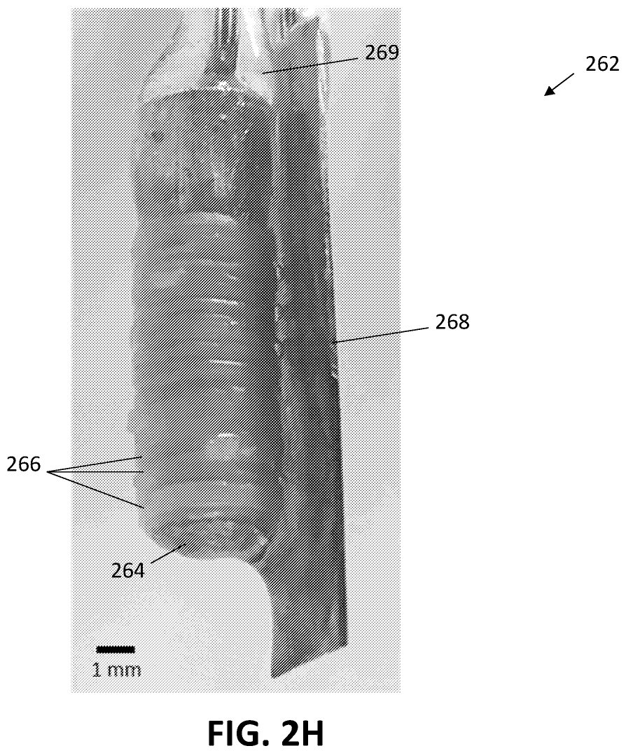

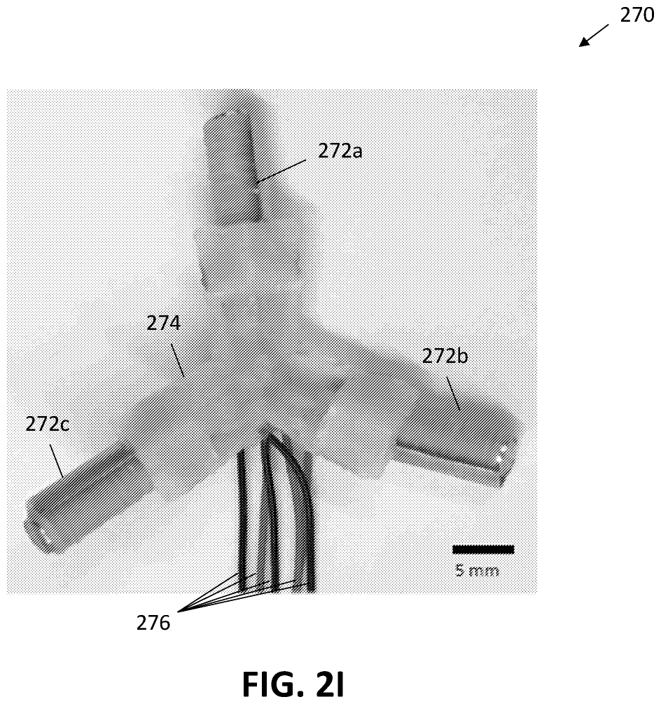

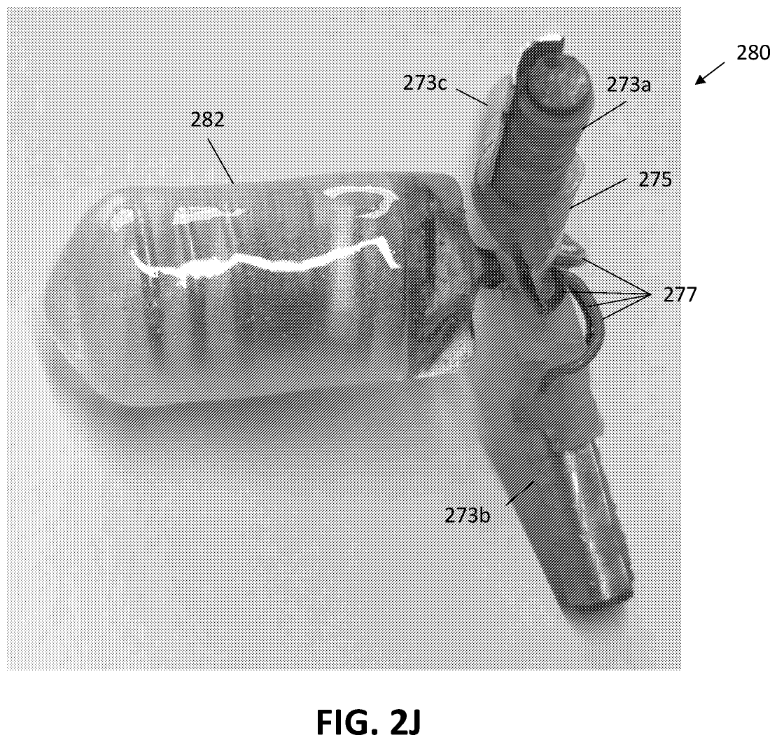

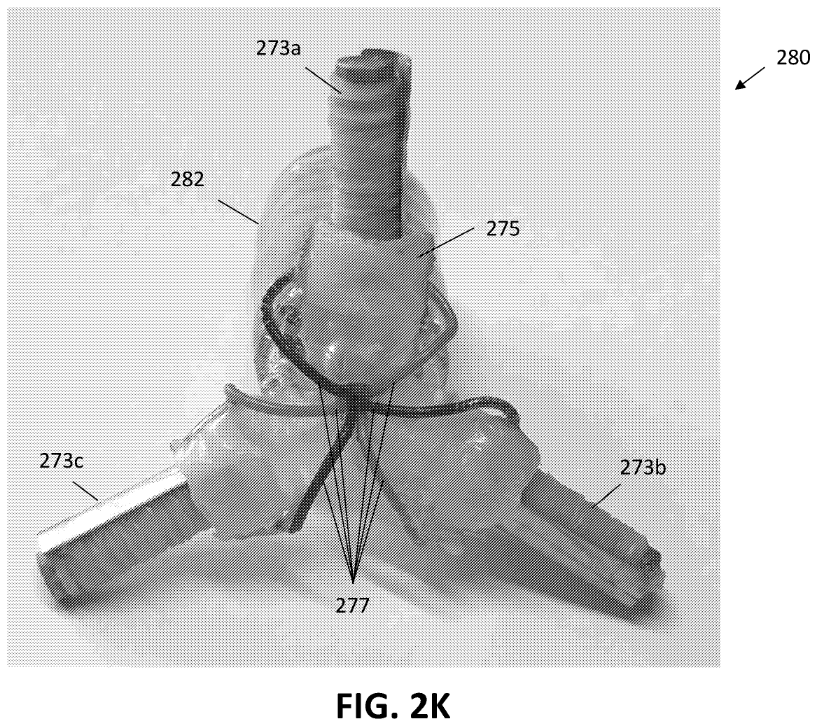

FIG. 2A shows a schematic of an orally-administered biomedical electronic device 230 powered by three CEH cells 232a-232c. The biomedical electronic device 230 includes a capsule 244 forming a sealed cavity. For administration to a human, the capsule is about 30 mm or less in length and about 13 mm or less in diameter. For example, the capsule may have a cylindrical shape with a diameter of 11 mm and a length of 22 mm. The capsule may be made of a biocompatible material and made be 3D printed.

Electronic components are disposed inside of and sealed within the cavity in the capsule 244. The CEH cells 232a-232c are electrically coupled to the energy harvesting circuit 250 (e.g., a DC/DC converter). The energy harvesting circuit 250 is electrically coupled to a circuit 252 for receiving signals from one or more sensors (e.g., temperature sensors or pH sensors). For example, sensors coupled to the sensor circuit 252 may monitor gastric health. The energy harvesting circuit 250 is also coupled to an energy storage component 254 (e.g., a rechargeable battery or capacitor) for storing energy produced by the CEH cells 232a-232c. The energy harvesting circuit 250 converts the low voltage provided by the CEH cells 252a-252c to a higher voltage and stores the converted energy in the energy storage component 254. The energy harvesting circuit 250 is also electrically coupled to a microprocessor 256 and a wireless communication component 258 (e.g., a 915 MHz transceiver or a 2.4 GHz Bluetooth transceiver). The microprocessor 256 may process signals received by the sensor circuit 252 and send and receive signals via the communication component 258. The microprocessor 256 may also be coupled to a drug delivery actuator 257 that administers a drug from a drug reservoir 259 disposed in the capsule 244. For example, the drug may be an antacid or proton pump inhibitor (PPI) that treats the symptoms of gastroesophageal reflex disease (GERD) or gastric ulcers. Wires that electrically couple each electrical component in the capsule are flexible.

The CEH cells 232a-232c are disposed outside of the capsule 244 but are electrically coupled to at least one electronic component within the capsule 244 via flexible wires 240. At least part of the surface of the anode in each of the CEH cells is exposed to physiological fluids within the body. Although FIG. 2A shows three CEH cells coupled to the biomedical electronic device 230, the number of CEH cell coupled to the biomedical electronic device may vary between 1 and 6, depending on the amount of power to be supplied to the biomedical electronic device.

CEH cell 232a includes an anode 234a and a cathode 238a. The anode 234a and the cathode 238a are each electrically coupled through an external circuit routed through the capsule. The external circuit includes wires connecting each electrode to the energy harvesting circuit 250. The anode 234a is zinc metal formed in a cylindrical shape. One base of the cylindrical anode 234a forms the exposed surface 235a of the anode 234a where the anode undergoes galvanic oxidation dissolution. Another base of the cylindrical anode 234a is sealed (e.g., with biocompatible epoxy so that it does not interact or react). The lateral surface of the cylindrical anode 234a is covered with a seal. The seal is a stack of modified O-rings 236a disposed along the length of the lateral surface of the cylindrical anode 234a. The O-rings 236a partially overlap one another along the length of the lateral surface of the anode 234a to create a seal. As the exposed surface 235a of the anode undergoes galvanic oxidation dissolution, the O-rings 236a consecutively detach from the cylindrical anode 234a so that the exposed surface 235a of the anode 234a remains substantially constant during the CEH cell's lifetime.

The cathode 238a is a curved or flat section of gold metal disposed adjacent to the lateral surface of the cylindrical anode 234a along the length of the lateral surface of the cylindrical anode 234a. The cathode and the anode are attached to a holder that provides a desired distance between the anode and the cathode. Because the cathode 238a is disposed along the length of the lateral surface of the cylindrical anode, the distance between the anode's exposed surface 235a and the cathode 238a remains substantially constant (e.g., about 500 μm to about 1 mm) as the anode dissolves and the lateral length shortens. A shorter distance between the anode and the cathode yields higher power and shorter cell lifetime. A larger cathode yields higher power. The cathode curves around about 180 degrees of the surface of the anode so that it does not block O-rings from disengaging from the anode. Alternatively, the cathode may completely surround the anode for higher power output.

FIG. 2B is a circuit schematic of the microprocessor disposed in the capsule 244 shown in FIG. 2A. The microprocessor circuit includes a ground connection 1, a 3.3 V connection 2, 12C serial connections 3 and 4, analog-to-digital (ADC) connections 5 and 6 for sensor signal processing, 2 SPI serial connections 7-9, and digital output pins 12 and 13.

FIG. 2C is a photograph of an electrical assembly 290 that may be powered by one or more CEH cells. The electrical assembly 290 includes an energy harvesting circuit 292, an energy storage component 294, a microprocessor 296, an RF (915 MHz) transceiver 298, and an antenna 299.

FIG. 2D shows an embodiment of one of the modified O-rings 236b in FIG. 2A that is used to seal the anode surface. In their unstretched state, the O-rings may have a diameter 50% to 90% (e.g., 50%, 60%, 70%, 78%, 85%, or 90%) of the diameter of the cylindrical anode. In a version, the O-rings have a thickness of about 100 μm to about 1 mm (e.g., 100 μm, 500 μm, or 750 μm). The O-rings are made of biocompatible elastomer. The cross-section of the O-ring 236b has a step-edge so that the section of the O-ring 236b with a smaller diameter 237 fits into the larger diameter section of a neighboring O-ring and another neighboring O-ring's smaller diameter section fits into the larger diameter section 239 of the O-ring 236b. The tolerance between O-rings is about 200 μm. The O-rings are stretchy so that they fit together tightly and fit tightly against the surface of the anode. The inner diameter of the larger diameter section 239 is greater than the outer diameter of the smaller diameter section 237 so that the O-rings disengage from the anode when not stretched to fit together. This configuration of partially overlapping O-rings that fit into one another creates a seal along the lateral surface of the cylindrical anode 234a better than conventional O-rings with round cross-sections. Conventional O-rings may be used to create the seal, but they have a high risk of leakage between O-rings that leads to less control of power output and lifetime. The modified O-rings create a cylindrical stack having a substantially smooth lateral surface, whereas a gap would exist between conventional O-rings. The modified O-rings may be fabricated via injection molding. FIG. 2E shows additional views of the modified O-ring 236b.

FIG. 2F shows other embodiments of modified O-rings 236 that may be used to seal the anode surface. Modified O-ring 236f has a groove on one side and a protrusion on the other side, where the shape of the groove is the same or similar to the shape of the protrusion (e.g., hemispherical, square, or rectangular). The protrusion of one O-ring fits into the groove of another O-ring to create the seal. Modified O-ring 236g has a larger diameter section and a smaller diameter section like modified O-ring 236b shown in FIG. 2D, except that the larger diameter section of modified O-ring 236g is not parallel to the smaller diameter section. Instead, the larger diameter section is at an oblique angle (e.g., acute or obtuse) with respect to the smaller diameter section. The angled larger diameter section creates a tight seal against the small diameter section of a neighboring O-ring.

FIG. 2G shows part of an embodiment of CEH cell 232d. The CEH cell 232d includes an anode 234d, a cathode 238d, and a plurality of modified O-rings 236d that form a seal around the lateral surface of the anode 234d. One base surface 235d of the cylindrical anode 234d is left uncovered to form an exposed anode surface where galvanic oxidation dissolution occurs. The dissolution rate of the anode is controlled by modifying the area of the exposed surface 235d of the anode. The lifetime and power of the CEH cell can be tuned by changing the geometry of the cylindrically shaped anode 234d. The anode 234d has a diameter less than 10 mm and a length less than 30 mm. A larger diameter of the cylindrically shaped anode 234d provides higher power output. Longer lengths of the cylindrically shaped anode 234d provide longer lifetimes. For example, a zinc anode 234d having a diameter of about 3.18 mm and a length of 10 mm had a lifetime of approximately 2 months and a power output of about 100 μW in simulated gastric fluid with a pH of 1 and a temperature of 37° C.

FIG. 2H shows a picture of CEH cell 262. The CEH cell 262 includes an anode 264, a cathode 268 and a plurality of modified O-rings 266 forming a seal around the lateral surface of the anode 264. The cathode 268 is a flat sheet of gold. The cathode and anode are held adjacent to one another with a holder 269 made of polymer and/or epoxy.

FIG. 2I shows a picture of a three-cell CEH device 270 that may be coupled with an orally administered biomedical electronic device. The CEH device 270 includes a first CEH cell 272a, a second CEH cell 272b, and a third CEH cell 272c. Each CEH cell in the CEH device 270 includes a cathode and an anode. Each anode has a cylindrical shape, where one base is exposed, and the lateral surface is sealed with a stack of modified O-rings to provide controlled anode dissolution during galvanic reaction. The three CEH cells are coupled together with a polymer casing 274 that covers part of each cell towards the base of the anode opposite the exposed surface. The polymer casing is made of a flexible material (e.g., Elastollan) so that the device 270 can be folded together for oral administration and passage through the esophagus before unfolding in the stomach. The CEH device 270 has a shape that provides gastric retention according to U.S. Pat. No. 10,182,985 filed Jun. 11, 2014, which is hereby incorporated by reference. Wires 276 are soldered and electrically coupled to each electrode in the CEH device 270 to provide power to an electronic device. The CEH cells can be electrically connected in series or in parallel. Connecting CEH cells electrically in series or in parallel may increase the voltage and/or power output.

FIGS. 2J and 2K show two different views of an orally-administered biomedical electronic device 280. The device includes three CEH cells 273a, 273b, and 273c similar to the three-cell CEH device shown in FIG. 2I. The three CEH cells are mechanically coupled together by a polymer casing 275 disposed around the ends of the cells opposite the exposed surface of the anode. Wires 277 electrically connect the CEH cells to one or more electrical components inside of the capsule 282. The CEH cells can be electrically connected in series or in parallel. The capsule 282 houses one or more electronic components powered by the CEH cells. The electronic components may include a microprocessor, wireless transceiver, sensors, drug delivery system, energy harvester, energy storage (e.g., capacitors and/or batteries), and the associated passive components and printed circuit boards that connect and hold the electronic components. The capsule 282 is made of a biocompatible polymer that may be made using 3D printing.

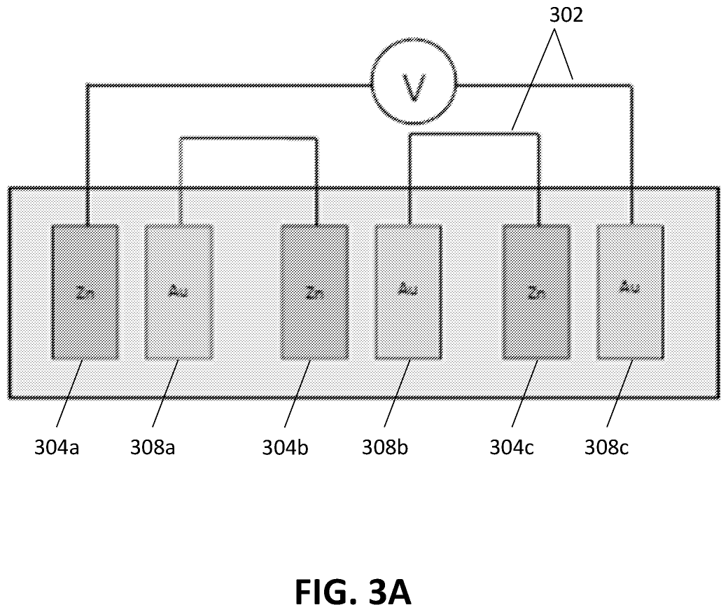

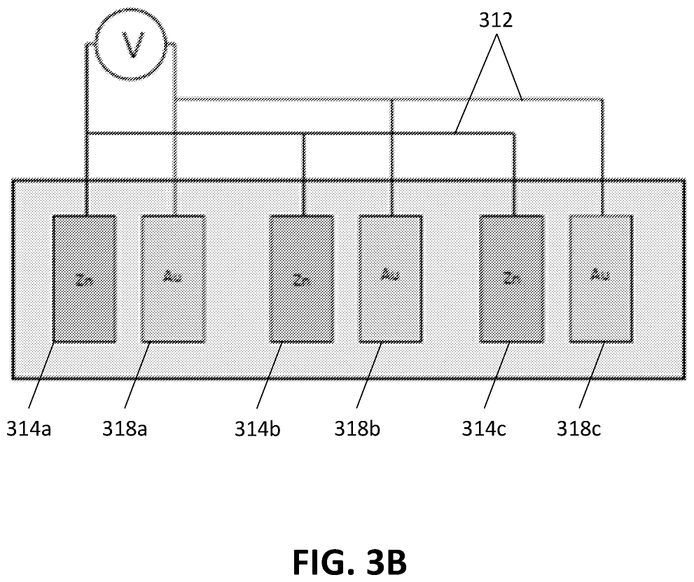

FIGS. 3A and 3B show diagrams of three galvanic cells electrically connected in series and in parallel, respectively. FIG. 3A includes zinc anodes 304a-304c and gold cathodes 308a-308c immersed in a liquid electrolyte. The electrodes in FIG. 3A are connected in series via wires 302. FIG. 3B includes zinc anodes 314a-314c and gold cathodes 318a-318c immersed in a liquid electrolyte. The electrodes in FIG. 3B are connected in parallel via wires 312. Connecting galvanic cells in series increases the voltage through the network. The total voltage is approximately equal to the sum of the voltages across each cell. Connecting galvanic cells in parallel increases the current through the network. The total current is approximately equal to the sum of the currents across each cell.

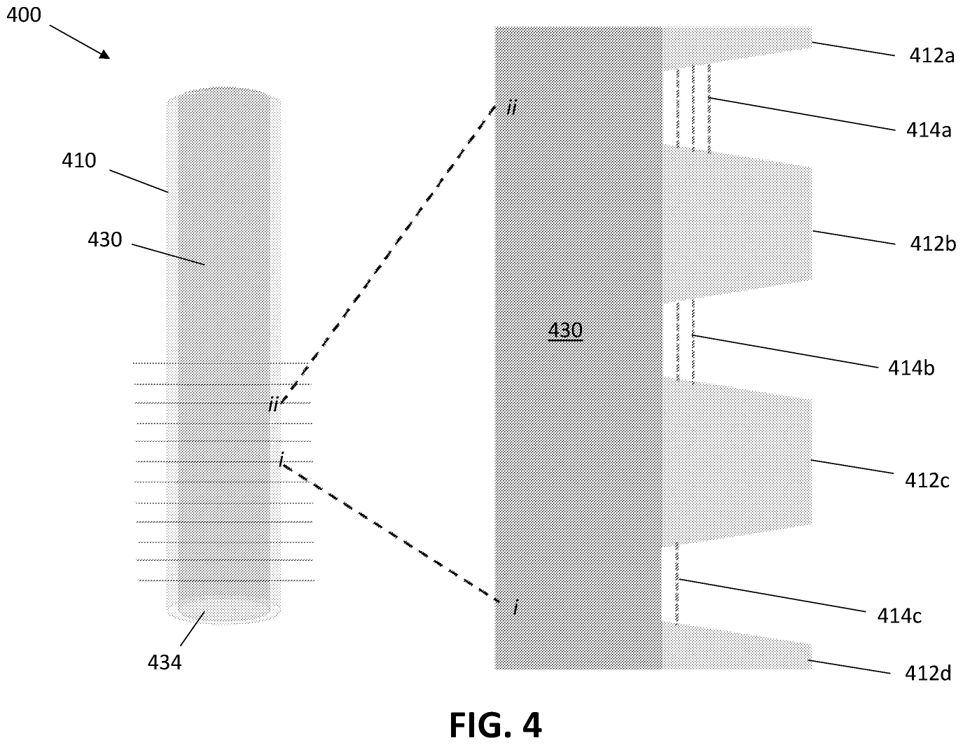

FIG. 4 shows another embodiment of a CEH cell anode structure 400 with a seal 410 formed around the lateral surface of the anode 430. FIG. 4 (left) shows the CEH cell anode structure 400 and FIG. 4 (right) shows a magnified view of part of the CEH cell anode structure 400. The anode 430 has a cylindrical shape with an exposed area 434 on one of the bases where galvanic oxidative dissolution occurs. The anode 430 has a diameter less than about 10 mm and a length less than about 30 mm. The exposed area 434 has a diameter less than about 10 mm. The seal 410 in this embodiment is formed of a stack of rings 412a-412d made of biocompatible elastomer (e.g., PDMS) or epoxy with a gap filler polymer 414a-414c disposed in the gaps between the rings 412a-412d. The rings are about 200 μm to about 500 μm wide and the gaps are about 10 μm to about 100 μm wide. The gap filler polymer 414a-414c is a biodegradable polymer (e.g., PCL, polyglycolide, polylactic acid, PLGA, or copolymers thereof). The dissolution rate of the gap filler polymer controlled by selecting the material and thickness of the gap filler polymer.

The stack of rings 412a-412d consecutively detaches as the exposed surface 434 of the anode 430 dissolves and the gap filler polymer 414a-414c dissolves. The thickness of the gap filler polymer 414a-414c progressively increases along the longitudinal axis of the anode 430 away from the exposed surface 434 of the anode 430. The progressively increasing thickness of the gap filler polymer 414a-414c provides progressively longer gap filler polymer degradation times. The gap filler polymer 414a-414c does not degrade sufficiently to release a ring from the stack of rings until the ring is near the exposed area 434 on the bottom flat surface of the anode. When the gap filler polymer in the gap between the last ring in the stack of rings and the second to last ring in the stack (nearest to the exposed surface of the anode 430) degrades sufficiently (e.g., having a thickness less than 500 nm), the last ring in the stack of rings is released from the stack of rings and disassociates from the CEH cell anode structure 400. In this way, the exposed area 434 of the anode 430 maintains a substantially constant surface area.

The seal 410 may be formed using a three-step process. The stack of rings 412a-412d may be made by coating the anode 430 with a thin layer of polymer via molding, dip coating, and/or spray coating processes. Once formed, the coating is cut using a blade or a laser cutter to form the stack of rings. Once the stack of rings is formed around the anode, the anode may be coated with a biodegradable polymer via dip coating and/or spray coating processes, forming the gap filler polymer in the gaps between the rings. The thickness of the gap filler polymer gradually increases along the longitudinal axis of the anode. The gradual increase in thickness is formed by applying additional polymer (e.g., additional coating layers) further from the expose surface 434.

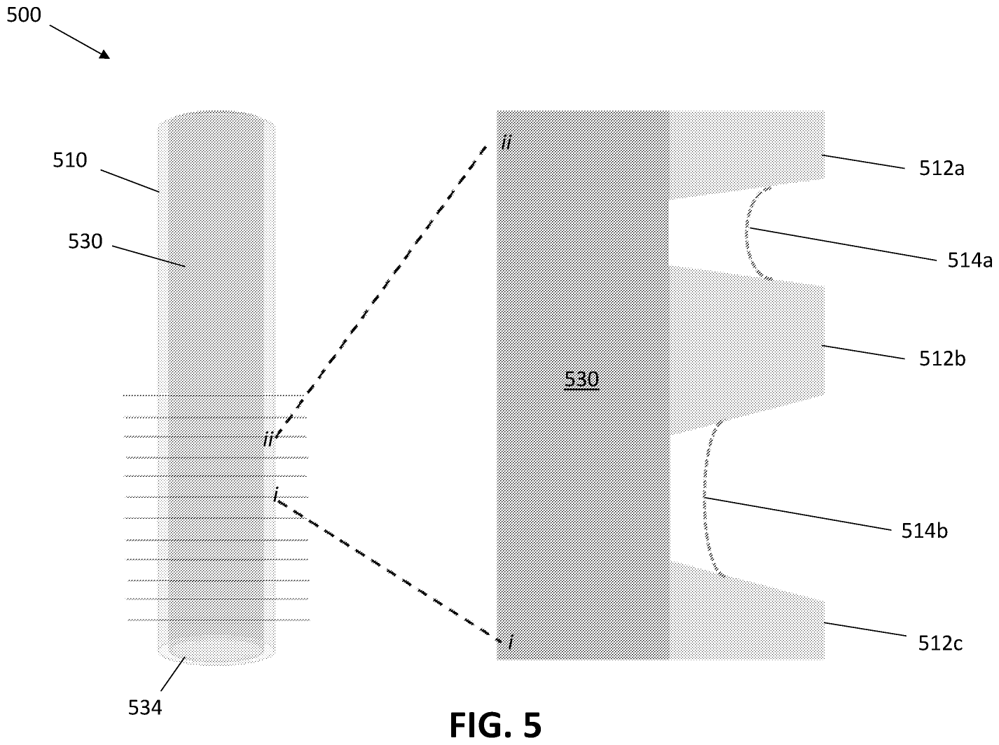

FIG. 5 shows another embodiment of a CEH cell anode structure 500 with a seal 510 formed around the lateral surface of the anode 530. FIG. 5 (left) shows the CEH cell anode structure 500 and FIG. 5 (right) shows a magnified view of part of the CEH cell anode structure 500. The anode 530 has a cylindrical shape with an exposed area 534 on one base where galvanic oxidative dissolution occurs. The seal 510 in this embodiment is formed of a stack of rings 512a-512c made of a biodegradable polymer (e.g., PCL, polyglycolide, polylactic acid, PLGA, or copolymers thereof). The rings are about 200 μm to about 500 μm wide. There is a gap between each ring in the stack of rings with a width of about 1 μm to about 100 μm, and the width of the gap gradually decreases along the longitudinal axis of the anode 530 towards the end opposite the exposed surface. A thinner layer of the same biodegradable polymer is disposed in the gaps between the rings, forming the gap filler polymer 514a, 514b. A cathode (not shown) is disposed on part of the lateral surface of the cylindrical anode structure 500 to form the CEH cell. The lifetime and power of the CEH cell can be tuned by changing the geometry of the cylindrical anode and the seal. During use, the stack of rings becomes consecutively detached as the cylindrical anode is dissolved and biodegradable polymer filler is degraded.

The stack of rings 512a-512c consecutively detaches as the exposed surface 534 of the anode 530 dissolves and as the ring polymer and the gap filler polymer dissolves. The thickness of the gap filler polymer 514a, 514b gradually increases along the longitudinal axis of the anode 530 towards the end opposite the exposed surface. The progressively increasing thickness of the gap filler polymer 514a, 514b provides progressively longer gap filler polymer degradation times. The thickness of the gap filler polymer is selected so that it degrades sufficiently to release the ring closest to the exposed surface 534 of the anode 530 as the anode 530 dissolves. The thickest portions of the gap filler polymer are furthest from the exposed surface 534 and have a thickness of about 200 μm to about 500 μm. The gap filler polymer 514a, 514b does not degrade sufficiently to release a ring from the stack of rings 512a-512c until the ring is near the exposed area 534 on the base of the anode 530. When the gap filler polymer in the gap between the last ring in the stack of rings and the second to last ring in the stack (nearest to the exposed surface 534 of the anode 530) degrades sufficiently (e.g., having a thickness less than 500 nm), the last ring in the stack of rings is released from the stack of rings and disassociates from the CEH cell anode structure 500. In this way, the exposed area 534 of the anode 530 maintains a substantially constant surface area.

The seal 510 may be formed using a three-step process. The stack of rings 512a-512c may be made by coating the anode 530 with a thin layer of biodegradable polymer via molding, dip coating, and/or spray coating processes. Once formed, the biodegradable polymer coating is cut using a blade or a laser cutter to form the stack of rings around the anode 530. Once the stack of rings is formed around the anode, the anode may be heated at a heating rate of 10° C. per minute to the melting point+10° C. of the biodegradable polymer and held at the melting point+10° C. until the biodegradable polymer partially melts. The amount of time the polymer coating is held at the elevated temperature depends on the gap width and the viscosity and flow speed of the biodegradable polymer. The polymer is melted so that it fills the gaps between rings. Because the gaps between rings gradually increases along the longitudinal axis of the anode further from the exposed base, the thickness of the melted polymer that fills the gaps gradually increases along the longitudinal axis of the anode away from the exposed end. For example, the melting point of PCL is about 60° C. and partially melting may mean that 0.2% to about 20% of the polymer filles the gaps between rings The anode 530 is then cooled to room temperature. A cathode is disposed on part of the lateral surface of the cylindrical anode structure 500 to form the CEH cell. The lifetime and power of the CEH cell can be tuned by changing the geometry of the cylindrical anode and the seal. During use, the stack of rings becomes consecutively detached as the cylindrical anode is dissolved and biodegradable polymer filler is degraded.

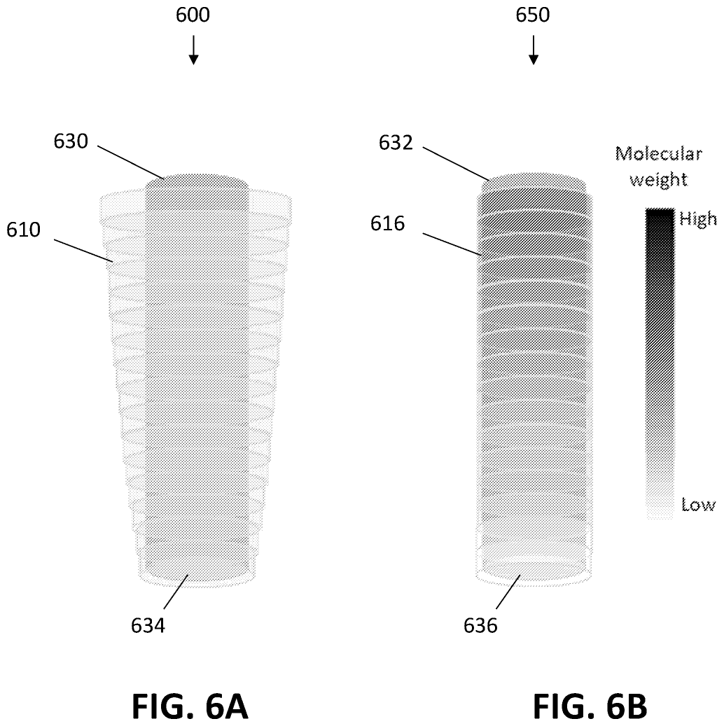

FIG. 6A shows another embodiment of a CEH cell anode structure 600 with a seal 610 formed around the lateral surface of the anode 630. The anode 630 has a cylindrical shape with an exposed area 634 on the bottom flat surface for galvanic oxidative dissolution. The seal 610 is formed of a biodegradable polymer (e.g., PCL, polyglycolide, polylactic acid, PLGA, or copolymers thereof). The seal 610 has a thickness gradient along the surface of the anode. The thickness of the polymer coating increases along the longitudinal axis of the anode 630 towards the end opposite the exposed surface 634. Because it is thicker, the polymer coating further from the exposed surface 634 of the anode 630 has a longer degradation time than the polymer coating towards the exposed end 634 of the anode. In this way the seal 610 progressively exposes the anode surface as the exposed anode surface 634 dissolves and the polymer coating degrades. The thickness gradient along the longitudinal axis of the anode 630 is tuned so that the polymer coating degradation rate is similar to the dissolution rate of the anode. The surface area of the exposed anode surface 634 remains substantially constant during operation. The thickness gradient may linearly increase along the longitudinal axis of the anode 630 so that the power output is more consistent. Alternatively, the thickness gradient may include step edges for ease of fabrication. The seal 610 may be formed via molding, dip coating, and/or spray coating processes. The thickness gradient is formed by increasing the number of coatings along the longitudinal axis of the anode towards the end opposite the exposed end 634.

FIG. 6B shows another embodiment of a CEH cell anode structure 650 with a seal 616 formed around the lateral surface of the anode 632. The anode 632 has a cylindrical shape with an exposed area 636 on one base where galvanic oxidative dissolution occurs. The seal 616 is formed of a biodegradable polymer. The seal 616 has a molecular weight gradient along the surface of the anode. The molecular weight of the polymer coating increases along the longitudinal axis of the anode 632 towards the base opposite the exposed surface 636. Because it has a higher molecular weight, the polymer coating further from the exposed base 636 of the anode 632 has a longer degradation time than the polymer coating towards the exposed base 636 of the anode. In this way the seal 616 progressively exposes the anode surface as the exposed anode surface 636 dissolves and the polymer coating degrades. The surface area of the exposed anode surface 636 remains substantially constant during operation. The molecular weight gradient along the longitudinal axis of the anode 632 is tuned so that the polymer coating degradation rate is similar to the dissolution rate of the anode. The molecular weight gradient may linearly increase along the longitudinal axis of the anode 630 so that the power output is more consistent. Alternatively, the molecular weight gradient may include step edges for ease of fabrication. The seal 616 may be formed via molding, dip coating, and/or spray coating processes. Polymers with different molecular weights may be applied to different sections along the longitudinal axis of the anode (e.g., via masking).

In another embodiment of a CEH cell anode structure, the seal around the anode's lateral surface is a biocompatible polymer that is brittle in acidic and/or neutral pH environments (e.g., in gastric fluid or intestinal fluid). For example, the biocompatible polymer in this embodiment may be shellac. As the exposed base of the cylindrical anode dissolves, a thin layer of polymer coating remains at the end of the anode without any mechanical support. The movement of the CEH cell within the body (e.g., via movement of the stomach) provides external force to the polymer coating that breaks the coating at the end of the anode and progressively exposes the anode surface. In this way the surface area of the exposed anode surface remains substantially constant during operation. The brittle coating may be applied via dip coating and/or spray coating.

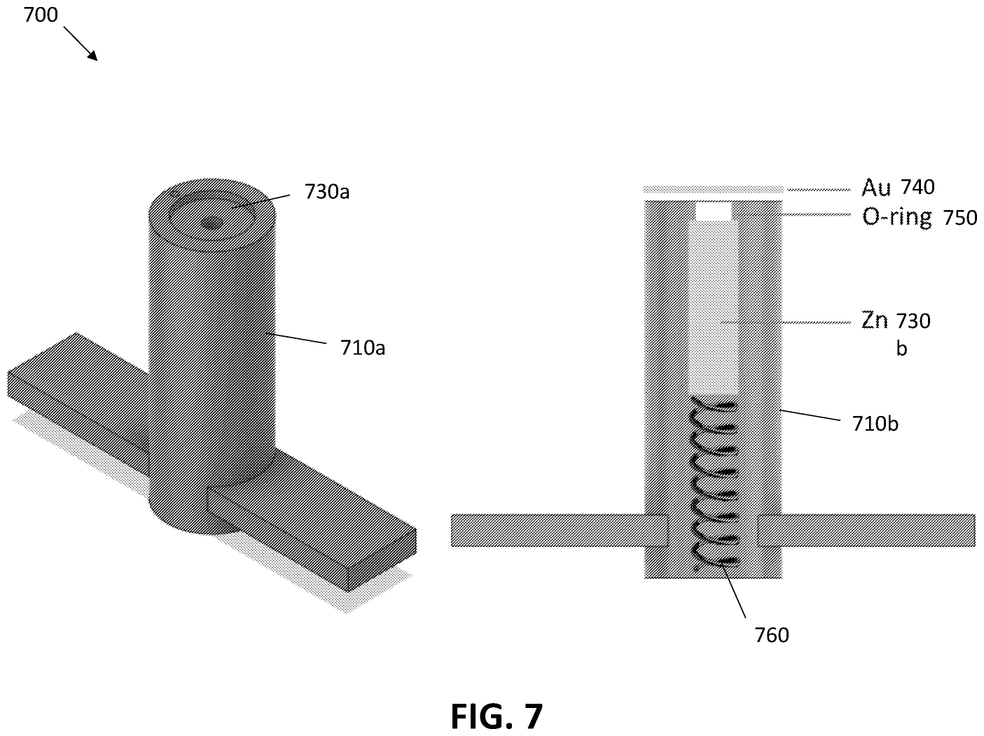

FIG. 7 shows another embodiment of a CEH cell 700. The CEH cell 700 includes an anode 730 disposed inside of a structure 710. The structure 710 is configured to expose a constant surface area of the anode 730 for galvanic oxidative dissolution. The structure has a partially open side so that at least part of a surface of the anode is exposed. The structure 710 has an outer diameter of less than 10 mm. For example, the structure 710 may be a hollow cylinder with an open end and a closed end. The anode 730 may be a solid cylinder disposed inside of the hollow cylinder with a first base of the anode 730 proximate to the open end of the structure 710, defining an exposed anode surface for galvanic oxidation dissolution. The anode 730 may have a diameter that is 100 μm to 500 μm smaller than the inner diameter of the structure 710. A gasket or O-ring may be disposed around the open end of the structure 710 in the space between the anode 730 and the open end of the structure 710 to create a seal in that space that prevents liquid from entering the cavity of the structure 710. The structure 710 may have a lip, overhang, or groove at its open end where the gasket or O-ring forms the seal. A mechanical spring 760 is disposed inside of the structure 710 to provide a pressure against the anode 730 to keep it in place and maintain the integrity of the gasket or O-ring seal as the exposed surface of the anode reacts and dissolves. The mechanical spring 760 may apply pressure to the second base of the anode. The mechanical spring 760 has a diameter that is less than or equal to the diameter of the anode 730 and a length in a neutral state that is the length of the anode 730. A cathode 740 is fixed to the outer surface of the structure 710 so that it is proximate to the exposed surface of the anode 730 and maintains a constant distance from the anode 730.

Any of the embodiments described above may be combined. The seal on the lateral surface of the anode may include aspects of any of the following types: the modified O-ring stack described in any of FIGS. 2A-2K, the ring stack described in FIG. 4, the ring stack described in FIG. 5, the thickness gradient polymer described in FIG. 6A, the molecular weight gradient polymer described in FIG. 6B, and/or the brittle polymer. Furthermore, any of the seals described above may be used in conjunction with the spring structure described in FIG. 7. Combining aspects of different types of seals and/or combining polymer seals with the spring structure described in FIG. 7 may provide a greater level of control over the anode dissolution rate, the power output, and the lifetime of the CEH cell. Any of the embodiments described above may be used to power any ingestible or implantable biomedical electronic device.

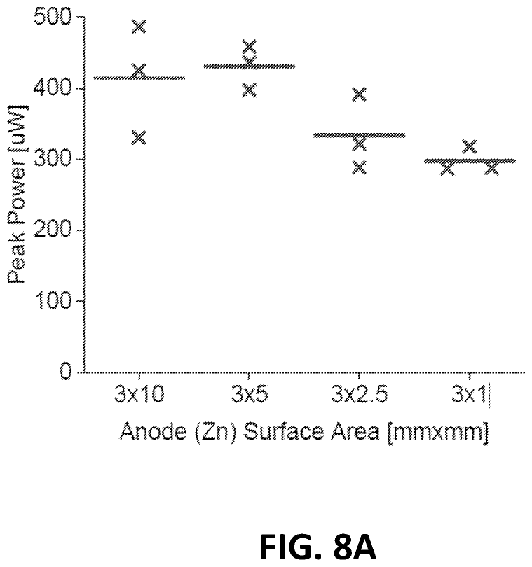

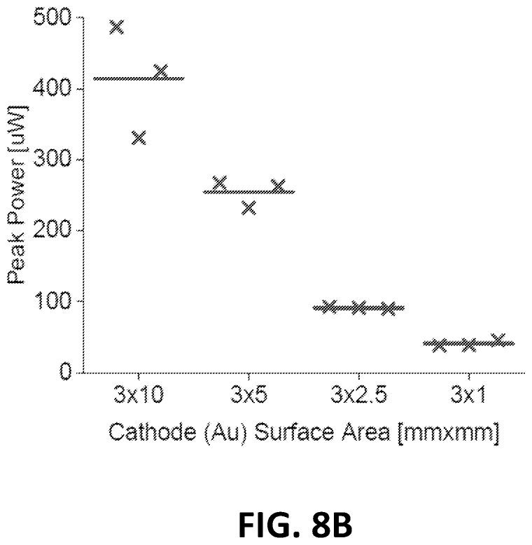

FIGS. 8A and 8B are in vitro plots of CEH cell power output vs. exposed anode surface area or cathode surface area, respectively. The CEH cell had a zinc anode and a gold cathode. The zinc anode used a stack of modified O-rings to create a seal, similar to that described above. The distance between anode and cathode was fixed at 3 mm for all experiments. Experiments were conducted in buffered simulated gastric fluid (SGF) having a pH of 1 at a temperature of 34° C.-37° C. In FIG. 8A, the anode surface area was varied while the cathode surface area was fixed at 3 mm×10 mm. In FIG. 8B, the cathode surface area was varied while the anode surface area was fixed at 3 mm×10 mm. The results showed that larger surface area electrodes provide higher peak power up to about 500 μW. Other results showed that a shorter distance (e.g., 500 μm) between electrodes also provides higher power.

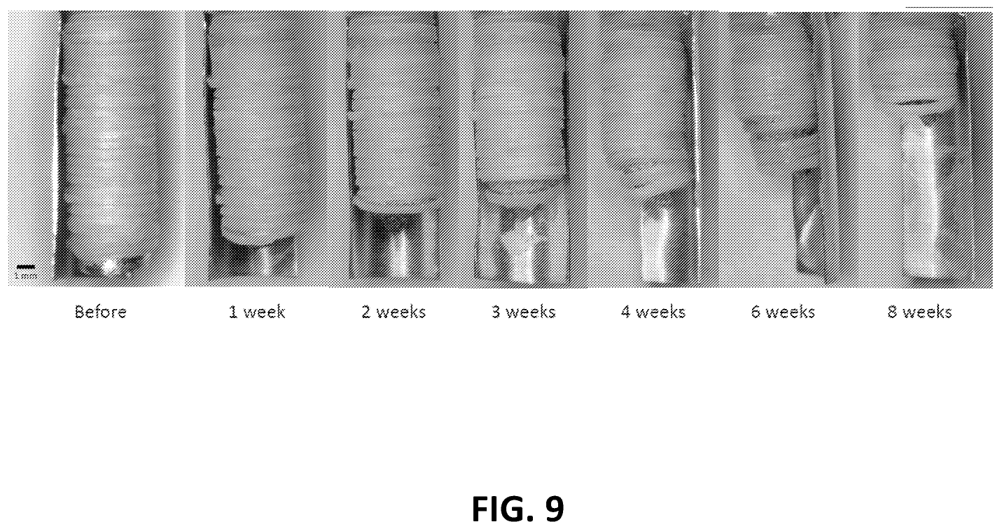

FIG. 9 shows in vitro changes in a CEH cell over time. The CEH cell had a zinc anode and a gold cathode. The zinc anode used a stack of modified O-rings to create a seal, similar to that described above. The zinc anode had a diameter of 3.18 mm. The gold cathode had dimensions of 5 mm×12.5 mm². The distance between anode and cathode was fixed at 500 μm as measured from the edge of the anode to the cathode. Experiments were conducted in buffered simulated gastric fluid (SGF) having a pH of 1 at a temperature of 34° C.-37° C. The electrodes were connected to a 1 kΩ resistor. Because of the specialized configuration of the CEH cell, which provides a constant anode surface area for electrochemical reaction, the CEH cell operated in vitro for 8 weeks.

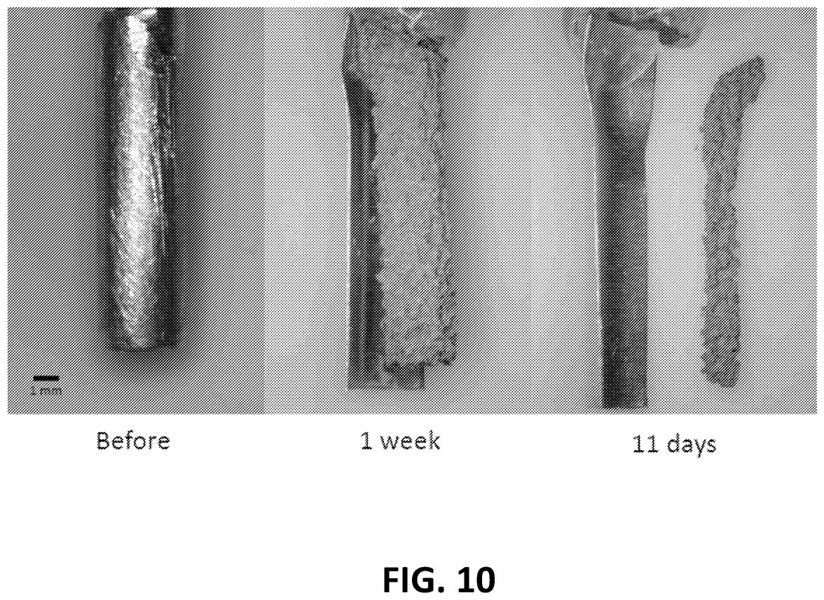

FIG. 10 shows in vitro changes in a conventional galvanic cell under the same conditions and having the same dimensions as the CEH cell but without any seal along the lateral surface of the anode that provides a controlled exposed anode surface area. As seen in the photographs of FIG. 10, the anode in the conventional cell was largely dissolved and detached from the CEH cell in 11 days. These results indicate that the CEH cell provides a substantially longer power output lifetime in comparison to a conventional galvanic cell.

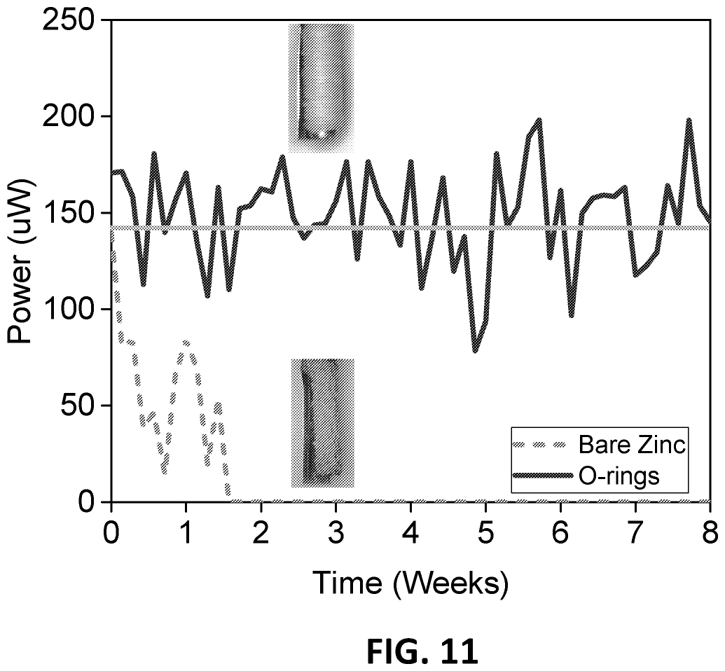

FIG. 11 shows in vitro measurements of power output by a CEH cell over a period of 8 weeks as compared to that of a conventional cell. In both cells, the anode was zinc having a diameter of 3.18 mm and a length of 12.5 mm, and the cathode was gold having dimensions 5 mm×12.5 mm². The CEH cell used modified O-rings to seal the lateral sides of the anode. The electrodes were connected to a 1 kΩ resistor. Experiments were conducted in buffered SGF having a pH of 1 at a temperature of 34° C.-37° C. The buffered SGF was replaced every 2 days during the experiment. In these experiments, the CEH cell provided an average power of 114 μW over 8 weeks. In comparison, the conventional cell stopped producing any power in less than 2 weeks. These results provide further indication that the CEH cell provides a substantially longer power output lifetime and a substantially steady power output in the range of 66 μW to 185 μW in comparison to a conventional galvanic cell.

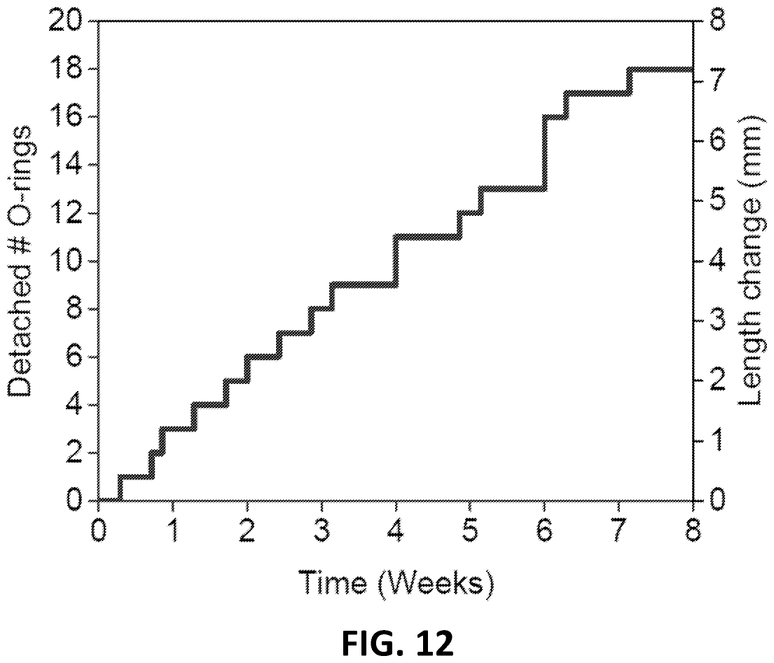

FIG. 12 shows in vitro measurements of modified O-ring detachment over time in a CEH cell using modified O-rings to seal the lateral surfaces of the anode. The anode was zinc having a diameter of 3.18 mm and a length of 12.5 mm, and the cathode was gold having dimensions 5 mm×12.5 mm². The electrodes were connected to a 1 kΩ resistor. Over 8 weeks, the CEH cell had an average mass change per day of about 8 mg, which is well below the maximum daily intake limit of zinc of 40 mg, as determined by the Food and Nutrition Board (FNB) at the Institute of Medicine of the National Academies. O-rings consecutively and steadily detached from the CEH cell at a rate of 2.25 rings per week over the 8-week period as the length of the anode decreased at a rate of 0.9 mm per week.

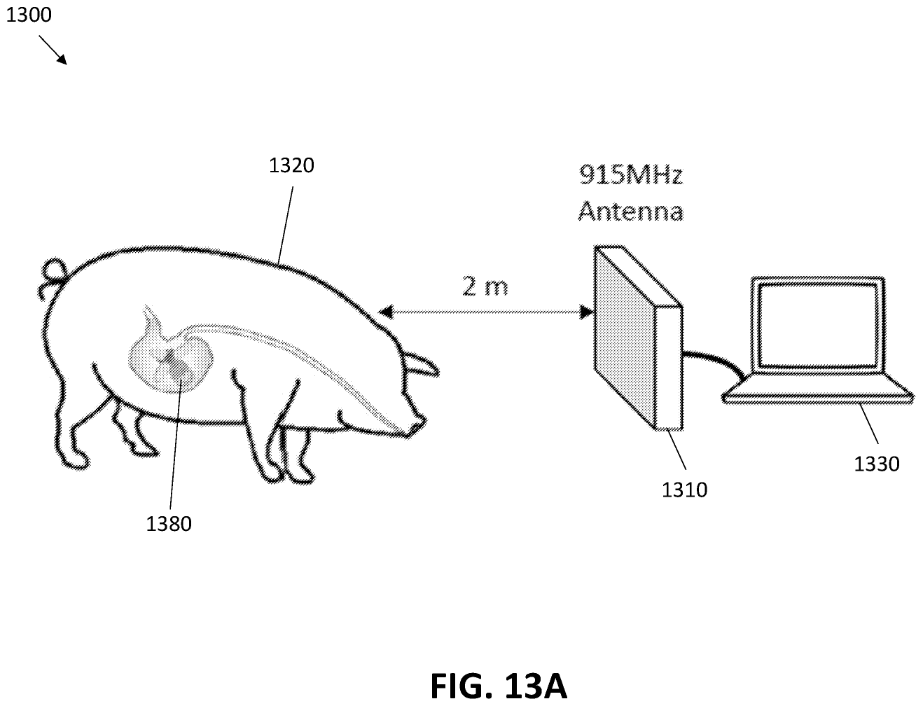

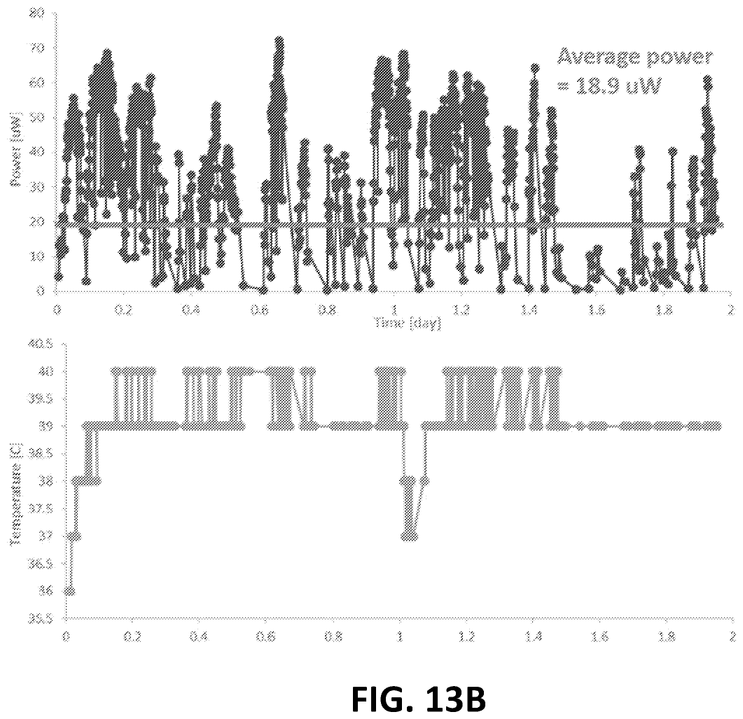

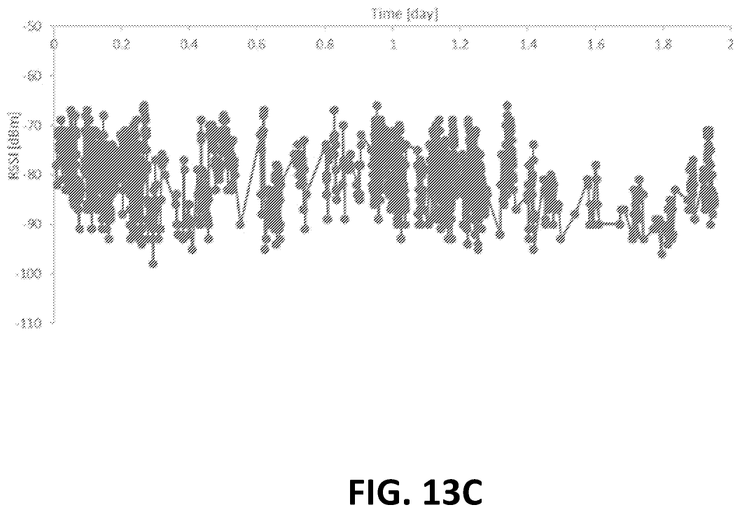

FIG. 13A shows the experimental setup in which a CEH device 1380 similar to that shown in FIGS. 2J-2K was studied in vivo using a swine model. After the CEH device 1380 was orally-administered to a pig 1320, a transmitter in the CEH device 1380 transmitted data signals to a 915 MHz antenna 1310 that was approximately 2 meters away from the pig 1320. The CEH device 1380 included a temperature sensor and a power output sensor and transmitted data from these two sensors to the antenna 1310. Data received by the antenna 1310 was transferred to a computer 1330, where it was analyzed and stored.

FIG. 13B shows power output by the CEH device (top) and the temperature inside the pig's GI tract as measured by the CEH device (bottom) over a two-day period. The average power output by the CEH device was 18.9 μW. FIG. 13C shows the received signal strength indicator (RSSI) by the antenna 1310 over the same time period. RSSI is a measurement of the power present in a received radio signal.

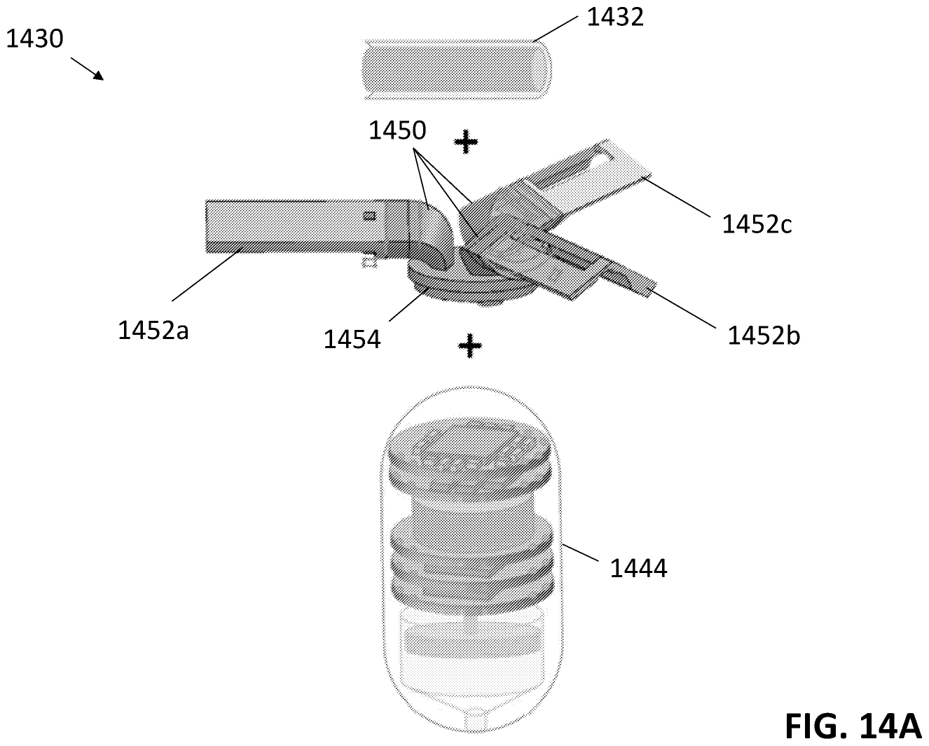

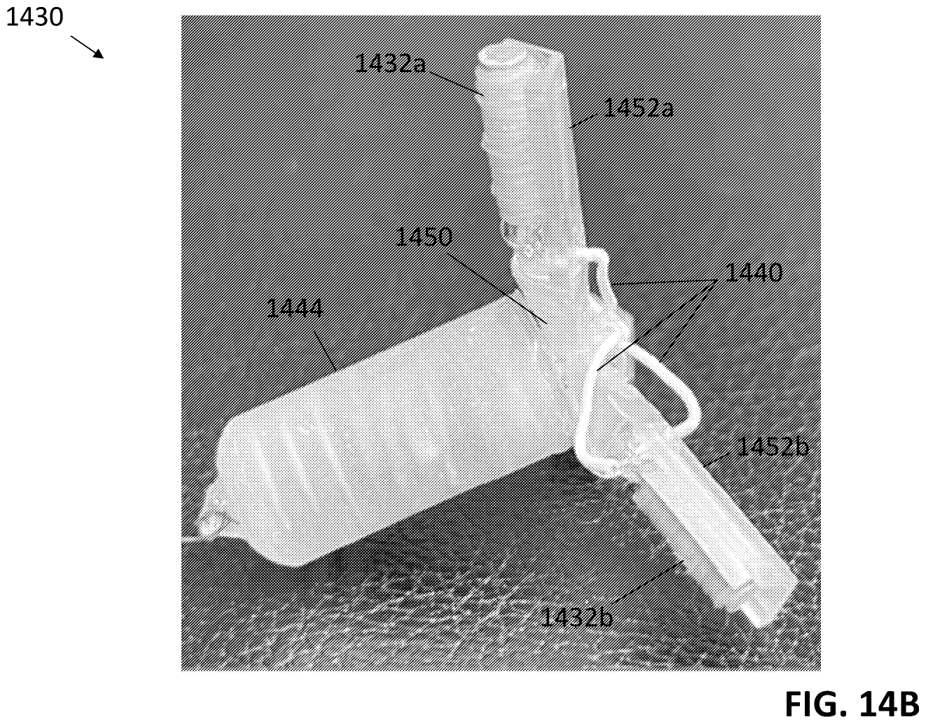

FIG. 14A shows an exploded view of an orally-administered biomedical electronic device 1430 and FIG. 14B shows a perspective view of the same device 1430. The biomedical electronic device 1430 is powered by three CEH cells 1432a-1432c, similar to those described above. The biomedical electronic device includes a capsule 1444 forming a sealed cavity. The capsule may be made of a biocompatible material and made be 3D printed. Electronic components are disposed inside of and sealed within the cavity in the capsule 244 and powered by the CEH cells 1432a-1432c. The CEH cells 1432a-1432c are electrically coupled to the electronic components in the capsule 1444 via flexible wires 1440, with two sets of wires per CEH cell.

The capsule 1444 is mechanically coupled to the CEH cells 1432a-1432c via a rigid disk 1454, a flexible core 1450, and support structures 1452a-1452c. The configuration of flexible core 1450 and support structures 1452a-1452c aids gastric residency and passage through the pylorus. The rigid disk 1454 is directly attached to the capsule 1444. The flexible core 1450 connects the rigid disk 1454 to the support structures 1452a-1452c. Each support structure supports a CEH cell. The flexible core 1450 has mechanical properties so that it is not too floppy and not too rigid. In other words, the mechanical properties of the flexible core 1450 are such that the force required to bend the flexible core 1450 is greater than 3 N. The flexible core 1450 may be made of elastollan. The flexible core 1450, the rigid disk 1454, and the support structures 1452a-1452c are made of materials that are compatible with gastric residency. The rigid disk 1454, the flexible core 1450, and the support structures 1452a-1452c may be manufactured using 3D printing.

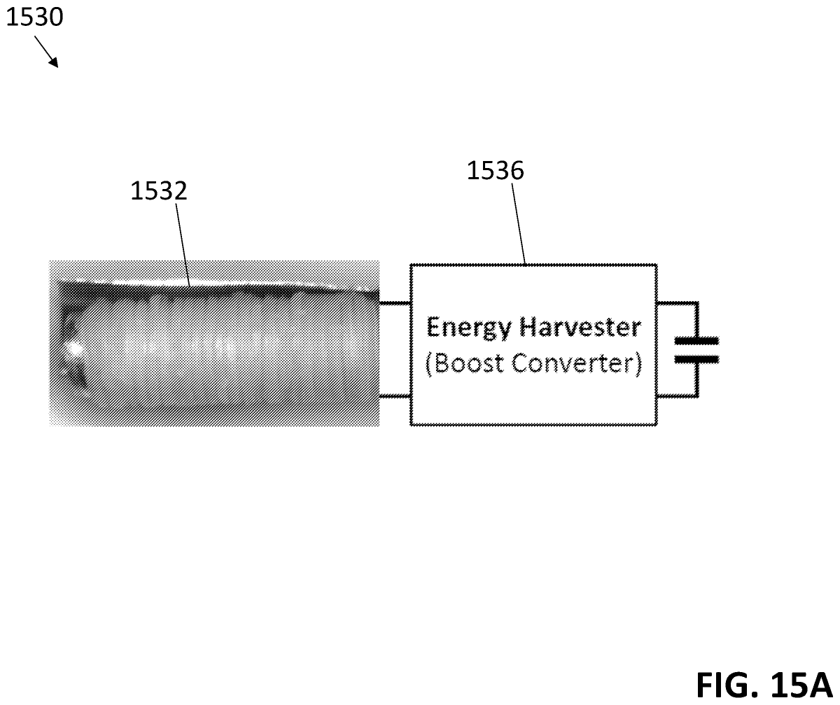

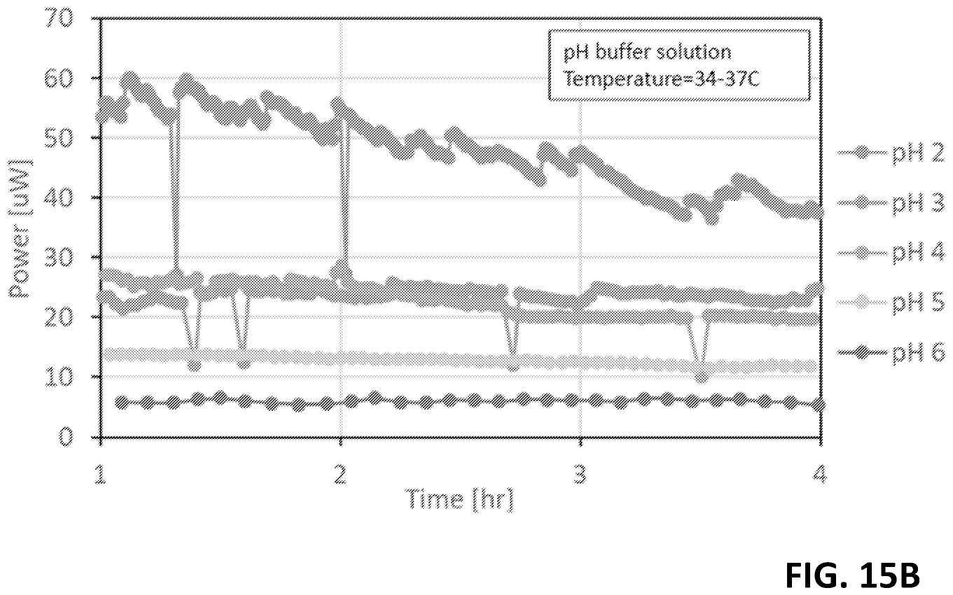

FIG. 15A shows a CEH cell 1532 electrically coupled with an energy harvester 1536 that together acts as a self-powered pH sensor 1530. The CEH cell 1532 is similar to those described above. The power output by the pH sensor 1530 scales with the pH of the solution in which the pH sensor 1530 is in. FIG. 15B is a graph showing power output by the pH sensor 1530 when the pH sensor 1530 is in different pH solutions with pH values of 2 to 6. The results show higher power output from the pH sensor 1530 when the sensor 1530 is in higher pH solutions.

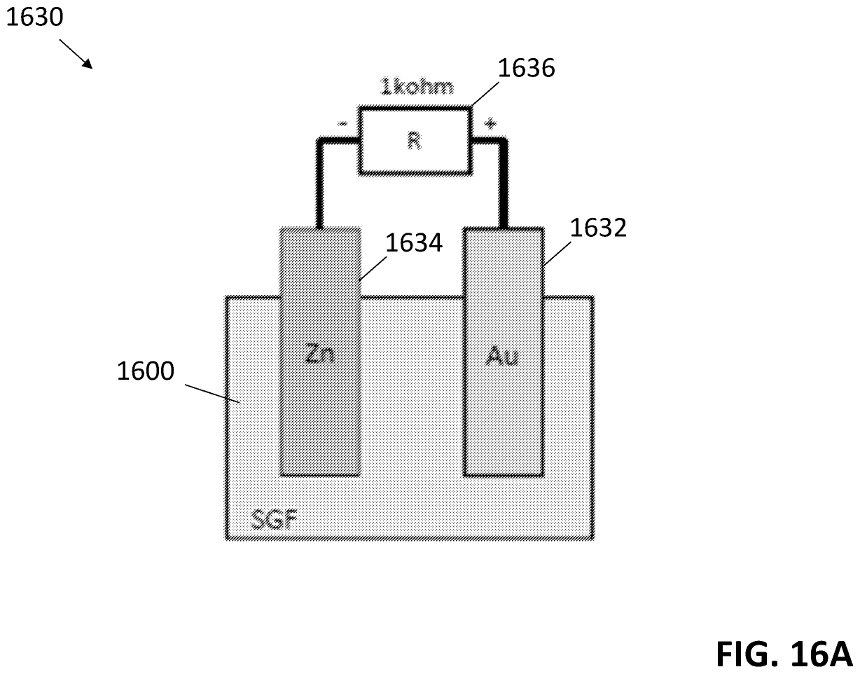

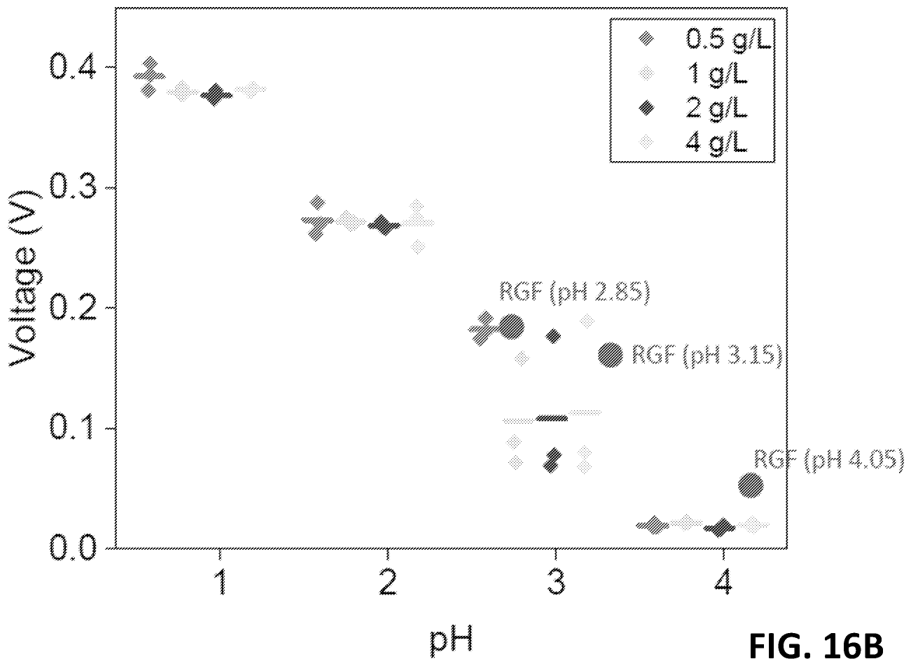

FIG. 16A shows another pH sensor 1630. The pH sensor 1630 includes a cathode 1632 and an anode 1634 electrically connected together via 1 kOhm resistor 1636. As an example, the cathode 1632 and the anode 1634 may both have column shapes with dimensions of about 3 mm diameter and about 12.5 mm height. The cathode 1632 and the anode 1634 may be held apart at a fixed distance of about 0.75 mm+0.25 mm. The cathode 1632 may be gold and the anode 1634 may be zinc. One or both of the electrodes 1632 and 1634 may optionally include any of the seals described above to prolong the sensor's operation time in acidic solutions. FIG. 16A shows the pH sensor 1630 disposed in simulated gastric fluid 1600. FIG. 16B is a graph showing voltage measured by the pH sensor 1630 in different simulated gastric fluid solutions having a pH of about 0.5 to about 4 and salt concentrations of about 0.5 g/L to about 4 g/L, and real gastric fluid (large circles) having a pH of about 0.5 to about 4. Data was collected at a temperature of about 34° C. to about 37° C., with three measurements per condition. The data in FIG. 16B shows that the voltage reading of the pH sensor 1630 negatively correlates with the pH of the solution in which the sensor 1630 is disposed. The pH sensor 1630 can thus be used to accurately measure the pH of SGF and RGF in which the sensor is disposed.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A device configured to be administered via an oral route by a subject, the device comprising:
an anode comprising a first metal;
a cathode comprising a second metal; and
a seal disposed on all surfaces of the anode except for a portion constituting an exposed surface of the anode;
wherein:
the cathode is disposed on at least part of the seal;
the cathode is electrically coupled to the anode;
the anode and the cathode are configured to provide DC power to the device when the exposed surface undergoes galvanic oxidation dissolution in at least one liquid or hydrogel; and
the seal is configured to incrementally detach from the anode when the exposed surface undergoes galvanic oxidation dissolution in the at least one liquid or hydrogel.

2. The device of claim 1, wherein the seal comprises a plurality of partially overlapping O-rings arranged to form a cylindrical stack having a substantially smooth lateral surface.

3. The device of claim 2, wherein the O-rings comprise an elastomer.

4. The device of claim 1, wherein the seal comprises:
a plurality of O-rings arranged adjacent to each other in a cylindrical stack, the stack having a gap between each adjacent O-ring; and
a biodegradable polymer disposed at least partially in the gap between each O-ring.

5. The device of claim 1, wherein the seal comprises a biodegradable polymer having at least one of:
a thickness gradient along a surface of the anode such that the seal is thicker along a longitudinal axis of the anode away from the exposed surface; or
a molecular weight gradient along a surface of the anode such that the molecular weight of the seal is higher along a longitudinal axis of the anode away from the exposed surface.

6. The device of claim 1, wherein the at least one liquid or hydrogel comprises at least one of gastric fluid or intestinal fluid.

7. The device of claim 1, further comprising an ingestible capsule having a cavity, the ingestible capsule comprising:
a microprocessor disposed in the cavity;
at least one sensor operably coupled to the microprocessor;
at least one actuator operably coupled to the microprocessor and the at least one sensor; and
an antenna operably coupled to the microprocessor.

8. The device of claim 1, wherein the first metal and the second metal are biocompatible.

9. The device of claim 8, wherein the first metal comprises zinc and the second metal comprises a noble metal.

10. The device of claim 1, wherein:
the anode has a cylindrical shape comprising a lateral surface, a first base, and a second base;
the exposed surface of the anode constitutes the first base;
the seal is disposed on both (a) the lateral surface and (b) the second base; and
the seal is configured to incrementally detach from the lateral surface along a longitudinal axis of the anode in a direction from the first base to the second base when the exposed surface undergoes galvanic oxidation dissolution in the at least one liquid or hydrogel.

11. The device of claim 1, wherein the exposed surface has a surface area that remains substantially constant during the galvanic oxidation dissolution.

12. The device of claim 1, wherein, once administered, the DC power is provided for about 8 days to about 60 days.

13. A system configured to be administered via an oral route by a subject, the system comprising:
a microprocessor;
at least one sensor operably coupled to the microprocessor;
at least one actuator operably coupled to the microprocessor and the at least one sensor;
an antenna operably coupled to the microprocessor; and
a galvanic cell providing power to the at least one sensor, the at least one actuator, and the microprocessor, the galvanic cell comprising:
an anode;
a seal disposed on all surfaces of the anode except for a portion constituting an exposed surface; and
a cathode electrically coupled to the anode and disposed on at least part of the seal;
wherein the seal is configured to maintain:
a substantially constant surface area of the exposed surface of the anode when the exposed surface undergoes galvanic oxidation dissolution in at least one liquid or hydrogel; and
a substantially constant distance between the cathode and the exposed surface of the anode when the exposed surface undergoes galvanic oxidation dissolution in the at least one liquid or hydrogel.

14. The system of claim 13, wherein galvanic cell is a first galvanic cell, and the system further comprises a second galvanic cell.

15. The system of claim 14, wherein the first galvanic cell and the second galvanic cell are electrically coupled in series.

16. The system of claim 14, wherein the first galvanic cell and the second galvanic cell are electrically coupled in parallel.

17. A method comprising:
orally administering a device to a subject, the device comprising:
an anode;
a seal disposed on all surfaces of the anode except for a portion constituting an exposed surface; and
a cathode disposed on at least part of the seal and electrically coupled to the anode;
wherein:
the anode and the cathode are configured to provide DC power to the device when the exposed surface undergoes galvanic oxidation dissolution in a liquid in the subject's gastrointestinal tract;
the exposed surface has a surface area that remains substantially constant when the exposed surface undergoes the galvanic oxidation dissolution; and
the cathode and the exposed surface are separated by a distance that remains substantially constant when the exposed surface undergoes the galvanic oxidation dissolution.

18. The method of claim 17, wherein:
the seal comprises a plurality of partially overlapping O-rings arranged to form a cylindrical stack having a substantially smooth lateral surface; and
the seal incrementally detaches from the anode as the O-rings consecutively detach from the stack when the exposed surface undergoes the galvanic oxidation dissolution.

19. The method of claim 17, wherein:
the seal comprises:
a plurality of O-rings arranged adjacent to each other in a cylindrical stack, the stack having a gap between each adjacent O-ring; and
a biodegradable polymer disposed at least partially in the gap between each O-ring;
and the seal incrementally detaches from the anode as the biodegradable polymer degrades, causing the O-rings to consecutively detach from the stack.

20. The method of claim 17, wherein, once administered, the DC power is provided for about 8 days to about 60 days.
